(19) United States
(12) Patent Application Publication
(10) Pub. No.: US 2020/0164008 A1
(43) Pub. Date: May 28, 2020

(54) APHERESIS METHODS AND USES

(71) Applicant: SPARK THERAPEUTICS, INC., Philadelphia, PA (US)

(72) Inventors: John Fraser WRIGHT, Princeton, NJ (US); Marcus Eugene CARR, JR., Holland, PA (US)

(73) Assignee: SPARK THERAPEUTICS, INC., Philadelphia, PA (US)

(21) Appl. No.: 16/631,692

(22) PCT Filed: Jul. 17, 2018
(86) PCT No.: PCT/US2018/042553

§ 371 Date: Jan. 16, 2020

Related U.S. Application Data
(60) Provisional application No. 62/533,579, filed on Jul. 17, 2017.

(51) Int. Cl. A61K 35/76; C12N 15/86; C07K 14/005; C07K 16/08; A61K 35/16; G01N 33/68; C12N 9/22; C12N 15/11; A61M 1/36; A61M 1/34; A61M 1/16

(52) U.S. Cl. CPC A61K 35/76; C12N 2800/80; C07K 14/005; C07K 16/081; A61K 35/16; G01N 33/6854; C12N 9/22; C12N 15/11; A61M 1/362; A61M 1/3496; A61M 1/1678; C12N 2750/14132; C12N 2750/14143; C12N 2750/14122; C12N 15/86

(57) ABSTRACT

Provided are methods of treating a subject in need of treatment for a disease caused by a loss of function or activity of a protein. Also provided are methods of treating a subject in need of treatment for a disease caused by a gain of function, activity or expression, of a protein.

### RELATED APPLICATIONS

This patent application claims the benefit of priority to U.S. patent application No. 62/533,579, filed Jul. 17, 2017. The entire contents of the foregoing application is incorporated herein by reference, including all text, tables, drawings and sequences.

### INTRODUCTION

Gene therapy (gene transfer) using recombinant adeno associated virus (rAAV) has shown promising potential to address unmet medical needs. For example, gene therapy using AAV expressing coagulation factor VIII and IX have shown promising safety and efficacy in human clinical trials (references).

AAV infection is common in the human population, and is not known to cause disease. The majority of human subjects who may benefit from AAV based gene therapy were previously infected by AAV. While infection can occur later in life, most frequently they occur in childhood. As with any viral infection, the host immune response to an AAV infection results in the formation of antibodies to AAV (AAV antibodies). After exposure to AAV, the time course of rapid AAV antibody formation (weeks), high titer antibody peaks attainment followed by the gradual decline in AAV antibody levels (years) is well known (references). Typical peak titers of AAV antibodies after AAV infection are >1:100, and can easily exceed 1:1000 (Calcedo et al, 2009).

In promising hemophilia clinical studies to date (George et al 2016, American Society for Hematology, San Diego Calif., Plenary Lecture; George et al 2017, International Society for Thrombosis and Hemostasis, Berlin, Germany), the best results have been observed following systemic (e.g. intravenous) administration of AAV vectors expressing the therapeutic transgene (FVIII or FIX) into human subjects who have no preexisting antibodies (titer <1:1). Good result may also be obtained in the case of very low titer of preexisting antibodies (1:1-1:2), and modest results may be obtained in the case of low titer preexisting antibodies (1:3-1:5). Levels of preexisting antibodies exceeding these levels correspond to marginal to poor gene transduction. The mechanism of this decline in gene transfer efficiency as a function of preexisting antibody titer is binding and neutralization of the AAV gene therapy vector by preexisting AAV antibodies. When bound by anti-AAV antibodies the vector is prevented from reaching and transducing target tissues and cells, such as cells of the liver including hepatocytes and endothelial cells that are the target of therapeutic gene transfer. Depending on the specific AAV serotype, up to and exceeding 50% of subjects in with hemophilia may be not eligible to benefit from AAV based gene therapy treatment because of preexisting AAV antibodies (Calcedo et al 2009).

### SUMMARY

Disclosed herein are methods and uses to remove, deplete, capture, and/or inactivate AAV antibodies in prospective mammals, such as human subjects who may benefit from AAV gene therapy. In certain aspects, AAV antibodies are present at levels that reduce or block therapeutic gene transfer vector transduction of target cells. In certain aspects, AAV antibodies are preexisting and may be present at levels that reduce or block therapeutic gene transfer vector transduction of target cells. In certain aspects, AAV antibodies may develop after exposure to AAV or administration of an AAV vector for gene therapy. If such antibodies develop after administration of an AAV vector for gene therapy, these subjects can also be treated in accordance with the invention.

The method is based on a medical device/procedure, commonly referred to as apheresis and in more particularly, plasmapheresis where blood products are involved. In certain embodiments, apheresis is used to benefit AAV gene therapy, particularly in a subject that has preexisting AAV antibodies or develops AAV antibodies after gene therapy.

In general terms, apheresis or plasmapheresis, is a process in which a human subject's plasma is circulated ex vivo (extracorporeal) through a device that modifies the plasma through addition, removal and/or replacement of components before its return to the patient. Plasmapheresis can be used to remove human immunoglobulins (e.g., IgG, IgE, IgA, IgD) from a blood product (e.g., plasma). This procedure depletes, captures, inactivates, reduces or removes immunoglobulins (antibodies) that bind AAV thereby reducing the titer of AAV antibodies in the treated subject thereby reducing the AAV antibodies that may contribute to AAV neutralization. A device useful in practicing the invention could in the form of an AAV capsid affinity matrix column. Passing blood product (e.g., plasma) of a human subject through an AAV capsid affinity matrix would result in binding only of AAV antibodies, and of all isotypes (including IgG, IgM, etc.).

A sufficient amount of plasmapheresis using a AAV capsid affinity matrix is predicted to substantially remove AAV capsid antibodies, and reduce the AAV capsid antibody titer (load) in the so treated human. In certain embodiments, titer in a treated subject is reduced substantially to low levels (to <1:5, or less, such as <1:4, or <1:3, or <1:2, or <1:1). A reduction in antibody titer will be temporary because the B lymphocytes that produce the AAV capsid antibodies would be expected to gradually cause the AAV capsid antibody titer to rebound to the steady state level prior to the plasmapheresis procedure intervention. Kinetics of this rebound based on the half-life of IgG (20 h) and that synthetic rate equals the decay rate for systems in steady state (corresponding to the steady state AAV capsid titer prior to the plasmapheresis method.

In the case where a pre-existing capsid antibody titer was reduced from 1:100 to 1:1, AAV antibody titer rebounds of approximately 0.15% (corresponding to a titer of 1:1.2) 0.43% (1:1.4), 0.9% (1:1.9), 1.7% (1:2.7), and 3.4% (1:4.4), occur at 1 hour, 3 hours, 6 hours, 12 hours and 24 hours, respectively, after completion of the plasmapheresis method. A temporary removal of AAV antibodies (e.g., that bind to AAV capsid) from such a subject would correspond to a window of time (for example, of about 24 hours or less, such as 12 hours or less, or 6 hours or less, or 3 hours or less, or 2 hours or less, or 1 hour or less) during which a therapeutic AAV vector could be administered to the subject and predicted to efficiently transduce target tissues without substantial neutralization of the AAV vector with the AAV antibodies.

In the case where a pre-existing capsid antibody titer was reduced from 1:1000 to 1:1, AAV antibody titer rebounds of approximately 0.15% (corresponding to a titer of 1:2.5) 0.4% (1:5.3), 0.9% (1:9.7), 1.7% (1:18), and 3.4% (1:35), occur at 1 hour, 3 hours, 6 hours, 12 hours and 24 hours, respectively, after completion of the plasmapheresis method. Thus, a window for administration of AAV vector will be comparatively shorter.

Parameters such as the type of AAV capsid affinity matrix can be varied according to the AAV antibody serotype(s) in a subject. Thus, an AAV capsid affinity matrix can be adjusted (increased or decreased) according to the AAV antibody serotype(s) in a subject. For example, if the antibodies bind to one more serotypes, such as AAV1, AAV2, AAV3, AAV4, AAV5, AAV6, AAV7, AAV8, AAV9, AAV10, Rh10, Rh74, SEQ ID NO:1 and SEQ ID NO:2 capsid protein(s), antibodies specific to one or more AAV1, AAV2, AAV3, AAV4, AAV5, AAV6, AAV7, AAV8, AAV9, AAV10, Rh10, Rh74, SEQ ID NO:1 and SEQ ID NO:2 capsid protein(s) can be used as the affinity matrix.

Parameters such as the amount of AAV capsid affinity matrix can be varied according to the AAV antibody titer in a subject. For example, the amount of AAV capsid affinity matrix can be adjusted (increased or decreased) according to the amount of AAV antibody in a subject. For high AAV antibody titers, amount of AAV capsid affinity matrix can be increased. For lower AAV antibody titers, amount of AAV capsid affinity matrix can be relatively less.

Parameters such as the amount of AAV capsid affinity matrix can also be varied according to volume of a blood product treated from a subject. For example, the amount of AAV capsid affinity matrix can be adjusted (increased or decreased) according to the volume of blood product to which the matrix is contacted.

In addition, the window of time after depletion, captures, inactivation or removal of AAV antibodies may vary depending on how fast AAV antibody rebounds. For example, in certain subjects AAV antibody rebound may be faster or slower. In the case of faster AAV antibody rebound, the window of time during which a therapeutic AAV vector could be administered to the subject will be comparatively shorter. In the case of slower AAV antibody rebound, the window of time during which a therapeutic AAV vector could be administered to the subject will be comparatively longer.

### DETAILED DESCRIPTION

AAV vectors possess a number of desirable features for such applications, including tropism for dividing and non-dividing cells. Early clinical experience with these vectors has demonstrated long-term expression in treated humans. In addition, early clinical trials have demonstrated no sustained toxicity and immune responses were minimal or undetectable. AAV are known to infect a wide variety of cell types in vivo and in vitro by receptor-mediated endocytosis or by transcytosis. These vector systems have been tested in humans targeting many tissues, such as, retinal epithelium, liver, skeletal muscle, airways, brain, joints and hematopoietic stem cells.

The invention provides compositions and methods for removing, depleting, capturing, and/or inactivating AAV binding antibodies. Such antibodies can be pre-existing in a subject such as a mammal, for example, human. Alternatively, such AAV binding antibodies can develop in a subject such as a mammal, for example, a human due to exposure to AAV or treatment/administration of the subject with an AAV vector. The invention compositions and methods employ an AAV binding antibody affinity matrix.

In some embodiments, AAV binding antibodies are removed, depleted, captured and/or inactivated from a blood product obtained from a subject by a process comprising apheresis. Non-limiting examples of apheresis include apheresis, plasmapheresis, cytapheresis or combinations thereof. Apheresis refers to a method for extracorporeal (ex vivo) manipulation, removal, depletion, and/or inactivation of components present in the blood or blood product of a subject. In some embodiments, following apheresis the blood or blood product is returned to a subject.

In a typical apheresis method, blood is obtained directly from a vein or artery of a subject. In some embodiments, the blood is separated into two or more blood products, a component (e.g., a cell or a protein) is removed from one of the blood products, the blood products are optionally combined. The blood is optionally returned directly back into the artery or vein of the patient.

More specifically, for example, in an apheresis method peripheral blood is removed from a subject by means of a suitable apheresis column or machine; anticoagulant agents are optionally added to the blood; the blood is separated into a cellular fraction (e.g., comprising red blood cells, white blood cells, and platelets) and a liquid fraction (e.g., plasma). The liquid fraction is then subjected to apheresis wherein a component (AAV binding antibodies) in the liquid fraction is removed, depleted, captured and/or inactivated. Next, the treated blood plasma can be combined together with the previously separated solid blood components and reinjected into the subject. Suitable methods and apparatuses for separating plasma from whole blood are known to those skilled in the art, for example, as described in U.S. Pat. No. 4,619,639, Any volume loss due to apheresis can be later replaced by a suitable solution, such as an isotonic saline solution.

In certain embodiments, an apheresis method removes, depletes, captures and/or and inactivates at least 20% to 50%, at least 55%, at least 60%, at least 65%, at least 70%, at least 75%, at least 80%, at least 85%, at least 90%, at least 95%, at least 98%, at least 99%, or 100% of the AAV binding antibodies from a blood product obtained from a subject. In certain embodiments, a method removes, depletes, captures and/or and inactivates at least 80%, at least 85%, at least 90%, at least 95%, at least 98%, at least 99%, or 100% of the AAV binding antibodies from a blood product obtained from a subject. Non-limiting examples of a blood product include whole blood, serum, plasma, the like, and a combination thereof. A blood product may be devoid of cells, or may include cells (e.g., red blood cells, platelets and/or lymphocytes).

Any AAV binding antibody affinity matrix can be attached to or immobilized on a substrate for manufacture of the compositions or for use in an apheresis method using any suitable method. For example, in one embodiment an antibody that binds to AAV binding antibodies can be attached to or immobilized on a substrate for an apheresis column or apheresis method as disclosed herein. In another embodiment, an AAV capsid protein or AAV capsid fragment can be attached to or mobilized on a substrate for an apheresis column or apheresis method as disclosed herein. Such AAV capsid proteins and fragments include VP1, VP2 and/or VP3 of any AAV serotype suitable for manufacture of the compositions or for use in an apheresis method.

The affinity matrix that binds to AAV antibodies can be immobilized on a substrate using any suitable method and any suitable substrate. In some embodiments, the affinity matrix immobilized on a substrate in an affinity column is suitable for apheresis applications. In some embodiments, an apherersis process comprises an apheresis method, device or column such as that disclosed in U.S. Pat. Nos. 9,726,666 and 8,877,177.

Substrates to which the affinity matrix that binds to AAV antibodies is immobilized thereto are typically solid substrates. A “solid substrate” refers to, for example, a material having a rigid or semi-rigid surface or surfaces, which may be regular or irregular in geometric configuration, and may take the form of beads, resins, gels, spheres, microspheres, particles, fibres or other geometric configurations or physical forms. A solid substrate typically comprises a material that is applicable in medical, biochemical or biological assays, for example, substrate used in apheresis, column chromatography for purification or separation of biological molecules or organic molecules and ELISA assays. Solid substrates may be porous or non-porous.

Solid substrates for immobilizing the affinity matrix that binds to AAV antibodies of the invention are known in the art. Nonlimiting examples of solid substrates include for example, polymers such as polysaccharides. Nonlimiting examples of polysaccharides are high molecular weight polysaccharides, in particular polysaccharides having a molecular weight of 100 kDa or more, such as agarose. Agarose may be in particulate form, which optionally can be cross-linked. A particular nonlimiting example of agarose is Sepharose™. Yet another nonlimiting example of a polysaccharide is cellulose, which optionally can be cross-linked.

Other polymers appropriate as a substrate include, for example, carboxylated polystyrene. Solid substrates may be provided in the form of magnetic beads. Glass is also an appropriate substrate material.

Any suitable blood or plasma filtration column or system can be adapted for an apheresis column or apheresis method disclosed herein. Non-limiting examples include columns described in U.S. Pat. No. 4,619,639, membrane filtration systems (e.g., MDF) used with suitable particles, surfaces, or substrates, and PlasmaFlo® OP-05(W)L and RheoFilter® AR2000 blood filters manufactured by Asahi Medical Company, Ltd. of Japan.

An apheresis column or method of the invention for removal, depletion, capturing and/or inactivating AAV binding antibodies from the blood of a subject can be carried out once or repeatedly, as needed, in order to achieve a desired result. In some embodiments, an apheresis method is performed daily, every other day, every 3^{rd} day, every 4^{th} day, once a week, biweekly, twice a month, once a month, every other month, or a combination thereof in an effort to obtain a beneficial therapeutic effect.

In certain embodiments, an AAV gene therapy vector described herein is administered after AAV binding antibodies are removed, depleted, captured and/or inactivated from a blood product of a subject. The AAV gene therapy vector can be administered to a subject immediately following an apheresis method. In some embodiments, an AAV gene therapy vector is administered within at least 1 minute, within at least 10 minutes, within at least 20 minutes, within at least 60 minutes, within at least 1 hour, within at least 4 hours, within at least 8 minutes, within at least 12 minutes, or within at least 24 hours after AAV binding antibodies are removed, depleted, captured and/or inactivated from the blood product of a subject. In some embodiments, an AAV gene therapy vector is administered within 1 minute to 24 hours, within 1 minute to 8 hours, or within 1 minute to 4 hours after AAV binding antibodies are removed, depleted, captured and/or inactivated from the blood product of a subject.

In certain embodiments, an AAV binding antibody affinity matrix comprises a covalent bond that couples the AAV binding antibody affinity matrix to the substrate. In some embodiments, a suitable covalent bond comprises a peptide bond.

In certain embodiments, an AAV binding antibody affinity matrix comprises a linker that couples the AAV binding antibody affinity matrix to the substrate. A linker can provide a mechanism for covalently attaching an AAV binding antibody affinity matrix to the substrate. Any suitable linker can be used in a composition or method disclosed herein. Any suitable covalent linkage or linker can be used to couple an AAV binding antibody affinity matrix to the substrate.

In some embodiments, a linker comprises one or more amino acids such as a peptide linker. A peptide linker may comprise any suitable number of amino acids. In some embodiments, a peptide linker comprises at least 1, at least 2, at least 3, at least 4, at least 5 or at least 10 amino acids. In certain embodiments, a peptide linker comprises 1 to 50, 1 to 20, 1 to 10, or 1 to 5 amino acids. In some embodiments, a peptide linker comprises 1, 2, 3, 4, 5, 6, 7, 8, 9 or 10 amino acids. Non-limiting examples of amino acid and peptide linkers include one or more glycine residues, one or more serine residues, or a combination thereof. Additional suitable linkers include one or more carbons, silanes, thiols, phosphonic acid, and polyethylene glycol (PEG), combinations thereof.

A covalent bond may be attached to the N-terminal or C-terminal of the AAV binding antibody affinity matrix. A linker may be attached to the N-terminal or C-terminal of the AAV binding antibody affinity matrix.

Methods of attaching two or more molecules using a covalent bond or linker are well known in the art and are sometimes referred to as “crosslinking.” Non-limiting examples of crosslinking include an amine reacting with a N-Hydroxysuccinimide (NHS) ester, an imidoester, a pentafluorophenyl (PFP) ester, a hydroxymethyl phosphine, an oxirane or any other carbonyl compound; a carboxyl reacting with a carbodiimide; a sulfhydryl reacting with a maleimide, a haloacetyl, a pyridyldisulfide, and/or a vinyl sulfone; an aldehyde reacting with a hydrazine; any non-selective group reacting with diazirine and/or aryl azide; a hydroxyl reacting with isocyanate; a hydroxylamine reacting with a carbonyl compound; and combinations thereof.

The term “vector” refers to small carrier nucleic acid molecule, a plasmid, virus (e.g., AAV vector), or other vehicle that can be manipulated by insertion or incorporation of a nucleic acid. Vectors can be used for genetic manipulation (i.e., “cloning vectors”), to introduce/transfer polynucleotides into cells, and to transcribe or translate the inserted polynucleotide in cells. An “expression vector” is a specialized vector that contains a gene or nucleic acid sequence with the necessary regulatory regions needed for expression in a host cell. A vector nucleic acid sequence generally contains at least an origin of replication for propagation in a cell and optionally additional elements, such as a heterologous polynucleotide sequence, expression control element (e.g., a promoter, enhancer), intron, an inverted terminal repeat (ITR), selectable marker (e.g., antibiotic resistance), polyadenylation signal.

A viral vector is derived from or based upon one or more nucleic acid elements that comprise a viral genome. A particular viral vector is an adeno-associated virus (AAV) vector.

The term “recombinant,” as a modifier of vector, such as recombinant AAV vector, as well as a modifier of sequences such as recombinant polynucleotides and polypeptides, means that the compositions have been manipulated (i.e., engineered) in a fashion that generally does not occur in nature. A particular example of a recombinant AAV vector would be where a nucleic acid sequence that is not normally present in the wild-type AAV genome is inserted within the AAV genome. Although the term “recombinant” is not always used herein in reference to AAV vectors, as well as sequences such as polynucleotides, recombinant forms including heterologous polynucleotides, are expressly included in spite of any such omission.

A “recombinant AAV vector” or “rAAV” is derived from the wild type genome of AAV by using molecular methods to remove the wild type genome from the AAV genome, and replacing with a non-native nucleic acid sequence, referred to as a heterologous nucleic acid. Typically, for AAV one or both inverted terminal repeat (ITR) sequences of AAV genome are retained. rAAV is distinguished from an AAV genome, since all or a part of the AAV genome has been replaced with a non-native sequence with respect to the AAV genomic nucleic acid. Incorporation of a non-native or heterologous sequence therefore defines the AAV vector as a “recombinant” vector, which can be referred to as a “rAAV vector.”

A rAAV sequence can be packaged-referred to herein as a “particle”—for subsequent infection (transduction) of a cell, ex vivo, in vitro or in vivo. Where a recombinant AAV vector sequence is encapsidated or packaged into an AAV particle, the particle can also be referred to as a “rAAV vector” or “rAAV particle.” Such rAAV particles include proteins that encapsidate or package the vector genome. In the case of AAV, they are referred to as capsid proteins.

An AAV vector “genome” refers to the portion of the recombinant plasmid sequence that is ultimately packaged or encapsidated to form a viral (e.g., AAV) particle. In cases where recombinant plasmids are used to construct or manufacture recombinant vectors, the vector genome does not include the portion of the “plasmid” that does not correspond to the vector genome sequence of the recombinant plasmid. This non vector genome portion of the recombinant plasmid is referred to as the “plasmid backbone,” which is important for cloning and amplification of the plasmid, a process that is needed for propagation and recombinant virus production, but is not itself packaged or encapsidated into virus (e.g., AAV) particles. Thus, a vector “genome” refers to the nucleic acid that is packaged or encapsidated by virus (e.g., AAV).

The terms “nucleic acid” and “polynucleotide” are used interchangeably herein to refer to all forms of nucleic acid, oligonucleotides, including deoxyribonucleic acid (DNA) and ribonucleic acid (RNA). Nucleic acids include include genomic DNA, cDNA and antisense DNA, and spliced or unspliced mRNA, rRNA tRNA and inhibitory DNA or RNA (RNAi, e.g., small or short hairpin (sh)RNA, microRNA (miRNA), small or short interfering (si)RNA, trans-splicing RNA, or antisense RNA). Nucleic acids include naturally occurring, synthetic, and intentionally modified or altered polynucleotides (e.g., variant nucleic acid).

Polynucleotides can be single, double, or triplex, linear or circular, and can be of any length. In discussing polynucleotides, a sequence or structure of a particular polynucleotide may be described herein according to the convention of providing the sequence in the 5′ to 3′ direction.

A “transgene” is used herein to conveniently refer to a heterologous nucleic acid that is intended or has been introduced into a cell or organism. Transgenes include any heterologous nucleic acid, such as a gene that encodes a polypeptide or protein or encodes an inhibitory RNA.

The term “transduce” and grammatical variations thereof refer to introduction of a molecule such as an rAAV vector into a cell or host organism. The heterologous nucleic acid/transgene may or may not be integrated into genomic nucleic acid of the recipient cell. The introduced heterologous nucleic acid may also exist in the recipient cell or host organism extrachromosomally, or only transiently.

A “transduced cell” is a cell into which the transgene has been introduced. Accordingly, a “transduced” cell (e.g., in a mammal, such as a cell or tissue or organ cell), means a genetic change in a cell following incorporation of an exogenous molecule, for example, a nucleic acid (e.g., a transgene) into the cell. Thus, a “transduced” cell is a cell into which, or a progeny thereof in which an exogenous nucleic acid has been introduced. The cell(s) can be propagated and the introduced protein expressed, or nucleic acid transcribed. For gene therapy uses and methods, a transduced cell or plurality of transduced cells can be in a subject.

An “expression control element” refers to nucleic acid sequence(s) that influence expression of an operably linked nucleic acid. Control elements, including expression control elements as set forth herein such as promoters and enhancers. Vector sequences including AAV vectors can include one or more “expression control elements.” Typically, such elements are included to facilitate proper heterologous polynucleotide transcription and if appropriate translation (e.g., a promoter, enhancer, splicing signal for introns, maintenance of the correct reading frame of the gene to permit in-frame translation of mRNA and, stop codons etc.). Such elements typically act in cis, referred to as a “cis acting” element, but may also act in trans.

Expression control can be effected at the level of transcription, translation, splicing, message stability, etc. Typically, an expression control element that modulates transcription is juxtaposed near the 5′ end (i.e., “upstream”) of a transcribed nucleic acid. Expression control elements can also be located at the 3′ end (i.e., “downstream”) of the transcribed sequence or within the transcript (e.g., in an intron). Expression control elements can be located adjacent to or at a distance away from the transcribed sequence (e.g., 1-10, 10-25, 25-50, 50-100, 100 to 500, or more nucleotides from the polynucleotide), even at considerable distances. Nevertheless, owing to the length limitations of AAV vectors, expression control elements will typically be within 1 to 1000 nucleotides from the transcribed nucleic acid.

Functionally, expression of operably linked nucleic acid is at least in part controllable by the element (e.g., promoter) such that the element modulates transcription of the nucleic acid and, as appropriate, translation of the transcript. A specific example of an expression control element is a promoter, which is usually located 5′ of the transcribed nucleic acid sequence. A promoter typically increases an amount expressed from operably linked nucleic acid as compared to an amount expressed when no promoter exists.

An “enhancer” as used herein can refer to a sequence that is located adjacent to the heterologous nucleic acid. Enhancer elements are typically located upstream of a promoter element but also function and can be located downstream of or within a sequence. Hence, an enhancer element can be located 10-50 base pairs, 50-100 base pairs, 100-200 base pairs, or 200-300 base pairs, or more base pairs upstream or downstream of a heterologous nucleic acid sequence. Enhancer elements typically increase expressed of an operably linked nucleic acid above expression afforded by a promoter element.

An expression construct may comprise regulatory elements which serve to drive expression in a particular cell or tissue type. Expression control elements (e.g., promoters) include those active in a particular tissue or cell type, referred to herein as a “tissue-specific expression control elements/promoters.” Tissue-specific expression control elements are typically active in specific cell or tissue (e.g., liver). Expression control elements are typically active in particular cells, tissues or organs because they are recognized by transcriptional activator proteins, or other regulators of transcription, that are unique to a specific cell, tissue or organ type. Such regulatory elements are known to those of skill in the art (see, e.g., Sambrook et al. (1989) and Ausubel et al. (1992)).

Incorporation of tissue specific regulatory elements in the expression constructs provides for at least partial tissue tropism for the expression of a heterologous nucleic acid encoding a protein or inhibitory RNA. Examples of promoters that are active in liver are the TTR promoter, human alpha 1-antitrypsin (hAAT) promoter; albumin, Miyatake, et al. J. Virol., 71:5124-32 (1997); hepatitis B virus core promoter, Sandig, et al., Gene Ther. 3:1002-9 (1996); alpha-fetoprotein (AFP), Arbuthnot, et al., Hum. Gene. Ther., 7:1503-14 (1996)], among others. An example of an enhancer active in liver is apolipoprotein E (apoE) HCR-1 and HCR-2 (Allan et al., J. Biol. Chem., 272:29113-19 (1997)).

Expression control elements also include ubiquitous or promiscuous promoters/enhancers which are capable of driving expression of a polynucleotide in many different cell types. Such elements include, but are not limited to the cytomegalovirus (CMV) immediate early promoter/enhancer sequences, the Rous sarcoma virus (RSV) promoter/enhancer sequences and the other viral promoters/enhancers active in a variety of mammalian cell types, or synthetic elements that are not present in nature (see, e.g., Boshart et al, Cell, 41:521-530 (1985)), the SV40 promoter, the dihydrofolate reductase promoter, the cytoplasmic β-actin promoter and the phosphoglycerol kinase (PGK) promoter.

Expression control elements also can confer expression in a manner that is regulatable, that is, a signal or stimuli increases or decreases expression of the operably linked heterologous polynucleotide. A regulatable element that increases expression of the operably linked polynucleotide in response to a signal or stimuli is also referred to as an “inducible element” (i.e., is induced by a signal). Particular examples include, but are not limited to, a hormone (e.g., steroid) inducible promoter. Typically, the amount of increase or decrease conferred by such elements is proportional to the amount of signal or stimuli present; the greater the amount of signal or stimuli, the greater the increase or decrease in expression. Particular non-limiting examples include zinc-inducible sheep metallothionine (MT) promoter; the steroid hormone-inducible mouse mammary tumor virus (MMTV) promoter; the T7 polymerase promoter system (WO 98/10088); the tetracycline-repressible system (Gossen, et al., Proc. Natl. Acad. Sci. USA, 89:5547-5551 (1992)); the tetracycline-inducible system (Gossen, et al., Science. 268:1766-1769 (1995); see also Harvey, et al., Curr. Opin. Chem. Biol. 2:512-518 (1998)); the RU486-inducible system (Wang, et al., Nat. Biotech. 15:239-243 (1997) and Wang, et al., Gene Ther. 4:432-441 (1997)]; and the rapamycin-inducible system (Magari, et al., J. Clin. Invest. 100:2865-2872 (1997); Rivera, et al., Nat. Medicine. 2:1028-1032 (1996)). Other regulatable control elements which may be useful in this context are those which are regulated by a specific physiological state, e.g., temperature, acute phase, development.

Expression control elements also include the native elements(s) for the heterologous polynucleotide. A native control element (e.g., promoter) may be used when it is desired that expression of the heterologous polynucleotide should mimic the native expression. The native element may be used when expression of the heterologous polynucleotide is to be regulated temporally or developmentally, or in a tissue-specific manner, or in response to specific transcriptional stimuli. Other native expression control elements, such as introns, polyadenylation sites or Kozak consensus sequences may also be used.

The term “operably linked” means that the regulatory sequences necessary for expression of a nucleic acid sequence are placed in the appropriate positions relative to the sequence so as to effect expression of the nucleic acid sequence. This same definition is sometimes applied to the arrangement of nucleic acid sequences and transcription control elements (e.g. promoters, enhancers, and termination elements) in an expression vector, e.g., rAAV vector.

In the example of an expression control element in operable linkage with a nucleic acid, the relationship is such that the control element modulates expression of the nucleic acid. More specifically, for example, two DNA sequences operably linked means that the two DNAs are arranged (cis or trans) in such a relationship that at least one of the DNA sequences is able to exert a physiological effect upon the other sequence.

Accordingly, additional elements for vectors include, without limitation, an expression control (e.g., promoter/enhancer) element, a transcription termination signal or stop codon, 5′ or 3′ untranslated regions (e.g., polyadenylation (polyA) sequences) which flank a sequence, such as one or more copies of an AAV ITR sequence, or an intron.

Further elements include, for example, filler or stuffer polynucleotide sequences, for example to improve packaging and reduce the presence of contaminating nucleic acid. AAV vectors typically accept inserts of DNA having a size range which is generally about 4 kb to about 5.2 kb, or slightly more. Thus, for shorter sequences, inclusion of a stuffer or filler in order to adjust the length to near or at the normal size of the virus genomic sequence acceptable for AAV vector packaging into virus particle. In various embodiments, a filler/stuffer nucleic acid sequence is an untranslated (non-protein encoding) segment of nucleic acid. For a nucleic acid sequence less than 4.7 Kb, the filler or stuffer polynucleotide sequence has a length that when combined (e.g., inserted into a vector) with the sequence has a total length between about 3.0-5.5 Kb, or between about 4.0-5.0 Kb, or between about 4.3-4.8 Kb.

The term “isolated,” when used as a modifier of a composition, means that the compositions are made by the hand of man or are separated, completely or at least in part, from their naturally occurring in vivo environment. Generally, isolated compositions are substantially free of one or more materials with which they normally associate with in nature, for example, one or more protein, nucleic acid, lipid, carbohydrate, cell membrane.

The term “isolated” does not exclude combinations produced by the hand of man, for example, a rAAV sequence, or rAAV particle that packages or encapsidates an AAV vector genome and a pharmaceutical formulation. The term “isolated” also does not exclude alternative physical forms of the composition, such as hybrids/chimeras, multimers/oligomers, modifications (e.g., phosphorylation, glycosylation, lipidation) or derivatized forms, or forms expressed in host cells produced by the hand of man.

The phrase “consisting essentially of” when referring to a particular nucleotide sequence or amino acid sequence means a sequence having the properties of a given reference sequence. For example, when used in reference to an amino acid sequence, the phrase includes the sequence per se and molecular modifications that would not affect the basic and novel characteristics of the sequence.

The term “identity,” “homology” and grammatical variations thereof, mean that two or more referenced entities are the same, when they are “aligned” sequences. Thus, by way of example, when two protein sequences are identical, they have the same amino acid sequence, at least within the referenced region or portion. Where two nucleic acid sequences are identical, they have the same nucleic acid sequence, at least within the referenced region or portion. The identity can be over a defined area (region or domain) of the sequence.

An “area” or “region” of identity refers to a portion of two or more referenced entities that are the same. Thus, where two protein or nucleic acid sequences are identical over one or more sequence areas or regions they share identity within that region. An “aligned” sequence refers to multiple protein (amino acid) or nucleic acid sequences, often containing corrections for missing or additional bases or amino acids (gaps) as compared to a reference sequence.

The identity can extend over the entire length or a portion of the sequence. In certain embodiments, the length of the sequence sharing the percent identity is 2, 3, 4, 5 or more contiguous amino acids or nucleic acids, e.g., 6, 7, 8, 9, 10, 11, 12, 13, 14, 15, 16, 17, 18, 19, 20, etc. contiguous nucleic acids or amino acids. In additional embodiments, the length of the sequence sharing identity is 21 or more contiguous amino acids or nucleic acids, e.g., 21, 22, 23, 24, 25, 26, 27, 28, 29, 30, 31, 32, 33, 34, 35, 36, 37, 38, 39, 40, etc. contiguous amino acids or nucleic acids. In further embodiments, the length of the sequence sharing identity is 41 or more contiguous amino acids or nucleic acids, e.g. 42, 43, 44, 45, 45, 47, 48, 49, 50, etc., contiguous amino acids or nucleic acids. In yet further embodiments, the length of the sequence sharing identity is 50 or more contiguous amino acids or nucleic acids, e.g., 50-55, 55-60, 60-65, 65-70, 70-75, 75-80, 80-85, 85-90, 90-95, 95-100, 100-150, 150-200, 200-250, 250-300, 300-500, 500-1,000, etc. contiguous amino acids or nucleic acids.

The extent of identity (homology) or “percent identity” between two sequences can be ascertained using a computer program and/or mathematical algorithm. For purposes of this invention comparisons of nucleic acid sequences are performed using the GCG Wisconsin Package version 9.1, available from the Genetics Computer Group in Madison, Wis. For convenience, the default parameters (gap creation penalty=12, gap extension penalty=4) specified by that program are intended for use herein to compare sequence identity. Alternately, the Blastn 2.0 program provided by the National Center for Biotechnology Information(found on the world wide web at ncbi.nlm.nih.gov/blast/; Altschul et al., 1990, J Mol Biol 215:403-410) using a gapped alignment with default parameters, may be used to determine the level of identity and similarity between nucleic acid sequences and amino acid sequences. For polypeptide sequence comparisons, a BLASTP algorithm is typically used in combination with a scoring matrix, such as PAM100, PAM 250, BLOSUM 62 or BLOSUM 50. FASTA (e.g., FASTA2 and FASTA3) and SSEARCH sequence comparison programs are also used to quantitate extent of identity (Pearson et al., Proc. Natl. Acad. Sci. USA 85:2444 (1988); Pearson, Methods Mol Biol. 132:185 (2000); and Smith et al., J. Mol. Biol. 147:195 (1981)). Programs for quantitating protein structural similarity using Delaunay-based topological mapping have also been developed (Bostick et al., Biochem Biophys Res Commun. 304:320 (2003)).

Nucleic acid molecules, expression vectors (e.g., AAV vector genomes), plasmids and heterologous nucleic acids may be prepared by using recombinant DNA technology methods. The availability of nucleotide sequence information enables preparation of isolated nucleic acid molecules of the invention by a variety of means. For example, nucleic acid sequences encoding a therapeutic protein can be made using various standard cloning, recombinant DNA technology, via cell expression or in vitro translation and chemical synthesis techniques. Purity of polynucleotides can be determined through sequencing, gel electrophoresis and the like. For example, nucleic acids can be isolated using hybridization or computer-based database screening techniques. Such techniques include, but are not limited to: (1) hybridization of genomic DNA or cDNA libraries with probes to detect homologous nucleotide sequences; (2) antibody screening to detect polypeptides having shared structural features, for example, using an expression library; (3) polymerase chain reaction (PCR) on genomic DNA or cDNA using primers capable of annealing to a nucleic acid sequence of interest; (4) computer searches of sequence databases for related sequences; and (5) differential screening of a subtracted nucleic acid library.

Nucleic acids may be maintained as DNA in any convenient cloning vector. For example, clones can be maintained in a plasmid cloning/expression vector, such as pBluescript (Stratagene, La Jolla, Calif.), which is propagated in a suitable E. coli host cell. Alternatively, nucleic acids may be maintained in vector suitable for expression in mammalian cells, for example, an AAV vector.

As disclosed herein, rAAV actors may optionally comprise regulatory elements necessary for expression of the heterologous nucleic acid in a cell positioned in such a manner as to permit expression of the encoded protein in the host cell. Such regulatory elements required for expression include, but are not limited to, promoter sequences, enhancer sequences and transcription initiation sequences as set forth herein and known to the skilled artisan.

Methods and uses of the invention include delivering (transducing) nucleic acid (transgene) into host cells, including dividing and/or non-dividing cells. The nucleic acids, rAAV vector, methods, uses and pharmaceutical formulations of the invention are additionally useful in a method of delivering, administering or providing sequence encoded by heterologous nucleic acid to a subject in need thereof, as a method of treatment. In this manner, the nucleic acid is transcribed and a protein or inhibitory nucleic acid may be produced in vivo in a subject. The subject may benefit from or be in need of the protein or inhibitory nucleic acid because the subject has a deficiency of the protein, or because production of the protein or inhibitory nucleic acid in the subject may impart some therapeutic effect, as a method of treatment or otherwise. For example, an inhibitory nucleic acid can reduce expression or transcription of an aberrant deleterious protein that is expressed in a subject in which the apparent or deleterious protein causes a disease or disorder, such as a neurological disease or disorder.

rAAV vectors comprising an AAV genome with a heterologous nucleic acid permit the treatment of genetic diseases. For deficiency state diseases, gene transfer can be used to bring a normal gene into affected tissues for replacement therapy, as well as to create animal models for the disease using antisense mutations. For unbalanced disease states, gene transfer could be used to create a disease state in a model system, which could then be used in efforts to counteract the disease state. The use of site-specific integration of nucleic acid sequences to correct defects is also possible.

In various embodiments, rAAV vectors comprising an AAV genome with a heterologous nucleic acid may be used, for example, as therapeutic and/or prophylactic agents (protein or nucleic acid). In particular embodiments, the heterologous nucleic acid encodes a protein that can modulate the blood coagulation cascade.

For example, an encoded FVIII or FVIII-BDD may have similar coagulation activity as wild-type FVIII, or altered coagulation activity compared to wild-type FVII. Administration of FVIII or FVIII-BDD-encoding rAAV vectors to a patient results in the expression of FVIII or FVIII-BDD protein which serves to normalize the coagulation cascade.

In additional embodiments, a heterologous nucleic acid encodes a protein (enzyme) that can inhibit or reduce the accumulation of glycogen, prevent the accumulation of glycogen or degrade glycogen. For example, an encoded GAA may have similar activity as wild-type GAA. Administration of GAA-encoding rAAV vectors to a patient with Pompe disease results in the expression of the GAA protein which serves to inhibit or reduce the accumulation of glycogen, prevent the accumulation of glycogen or degrade glycogen, which in turn can reduce or decrease one or more adverse effects of Pompe disease.

rAAV vectors may be administered alone, or in combination with other molecules. According to the invention, rAAV vectors or a combination of therapeutic agents may be administered to the patient alone or in a pharmaceutically acceptable or biologically compatible compositions.

Direct delivery of rAAV vectors or ex-vivo transduction of human cells followed by infusion into the body will result in expression of the heterologous nucleic acid thereby exerting a beneficial therapeutic effect on hemostasis. In the context of blood coagulation factor, such as Factor VIII, administration enhances pro-coagulation activity. In the context of an enzyme, such as GAA, administration reduces the amount or accumulation of glycogen, prevents accumulation of glycogen or degrades glycogen. This, in turn, can reduce or decrease one or more adverse effects of Pompe disease such as promoting or improving muscle tone and/or muscle strength and/or reducing or decreasing enlarged liver.

Recombinant AAV vector, as well as methods and uses thereof, include any viral strain or serotype. As a non-limiting example, a recombinant AAV vector can be based upon any AAV genome, such as AAV-1, -2, -3, -4, -5, -6, -7, -8, -9, -10, -11, -12, -rh74, -rh10 or AAV-2i8, for example. Such vectors can be based on the same strain or serotype (or subgroup or variant), or be different from each other. As a non-limiting example, a recombinant AAV vector based upon a particular serotype genome can be identical to the serotype of the capsid proteins that package the vector. In addition, a recombinant AAV vector genome can be based upon an AAV serotype genome distinct from the serotype of the AAV capsid proteins that package the vector. For example, the AAV vector genome can be based upon AAV2, whereas at least one of the three capsid proteins could be a SEQ ID NO:1, SEQ ID NO:2, AAV1, AAV3, AAV4, AAV5, AAV6, AAV7, AAV8, AAV9, AAV10, AAV11, AAV12, Rh10, Rh74 or AAV-2i8 or variant thereof, for example.

In particular embodiments, adeno-associated virus (AAV) vectors include SEQ ID NO:1, SEQ ID NO:2, AAV1, AAV2, AAV3, AAV4, AAV5, AAV6, AAV7, AAV8, AAV9, AAV10, AAV11, AAV12, Rh10, Rh74 and AAV-2i8, as well as variants (e.g., capsid variants, such as amino acid insertions, additions, substitutions and deletions) thereof, for example, as set forth in WO 2013/158879 (International Application PCT/US2013/037170), WO 2015/013313 (International Application PCT/US2014/047670) and US 2013/0059732 (U.S. Pat. No. 9,169,299, discloses LK01, LK02, LK03, etc.).

As used herein, the term “serotype” is a distinction used to refer to an AAV having a capsid that is serologically distinct from other AAV serotypes. Serologic distinctiveness is determined on the basis of the lack of cross-reactivity between antibodies to one AAV as compared to another AAV. Such cross-reactivity differences are usually due to differences in capsid protein sequences/antigenic determinants (e.g., due to VP1, VP2, and/or VP3 sequence differences of AAV serotypes). Despite the possibility that AAV variants may not be serologically distinct from a reference AAV or other AAV serotype, they differ by at least one nucleotide or amino acid residue compared to the reference or other AAV serotype.

Under the traditional definition, a serotype means that the virus of interest has been tested against serum specific for all existing and characterized serotypes for neutralizing activity and no antibodies have been found that neutralize the virus of interest. As more naturally occurring virus isolates of are discovered and/or capsid mutants generated, there may or may not be serological differences with any of the currently existing serotypes. Thus, in cases where the new virus (e.g., AAV) has no serological difference, this new virus (e.g., AAV) would be a subgroup or variant of the corresponding serotype. In many cases, serology testing for neutralizing activity has yet to be performed on mutant viruses with capsid sequence modifications to determine if they are of another serotype according to the traditional definition of serotype. Accordingly, for the sake of convenience and to avoid repetition, the term “serotype” broadly refers to both serologically distinct viruses (e.g., AAV) as well as viruses (e.g., AAV) that are not serologically distinct that may be within a subgroup or a variant of a given serotype.

As set forth herein, AAV capsid proteins and nucleic acids encoding the capsid proteins exhibit less than 100% sequence identity to a reference or parental AAV serotype such as SEQ ID NO:1, SEQ ID NO:2, AAV1, AAV2, AAV3, AAV4, AAV5, AAV6, AAV7, AAV8, AAV9, AAV10, AAV11, AAV12, Rh10, or AAV-2i8, but are distinct from and not identical to known AAV genes or proteins, such as SEQ ID NO:1, SEQ ID NO:2, AAV1, AAV2, AAV3, AAV4, AAV5, AAV6, AAV7, AAV8, AAV9, AAV10, AAV11, AAV12, Rh10, Rh74 or AAV-2i8. In one embodiment, an AAV capsid protein includes or consists of a sequence at least 80%, 85%, 85%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 99.9% identical to a reference or parental AAV capsid protein, such as SEQ ID NO:1, SEQ ID NO:2, AAV1, AAV2, AAV3, AAV4, AAV5, AAV6, AAV7, AAV8, AAV9, AAV10, AAV11, AAV12, Rh10, Rh74 or AAV-2i8.

In certain embodiments, a modified AAV capsid protein has 1, 2, 3, 4, 5, 5-10, 10-15, 15-20 or more amino acid substitutions. In certain embodiments, a modified AAV capsid protein has a peptide insertion length of 2, 3, 4, 5, 5-10, 10-15, 15-20, 20-25, 25-30, 30-35, 35-40, 40-50 or 50-60 amino acids.

rAAV vectors may be administered to a patient via infusion in a biologically compatible carrier, for example, via intravenous injection. The rAAV vectors may optionally be encapsulated into liposomes or mixed with other phospholipids or micelles to increase stability of the molecule.

rAAV vectors may be administered alone or in combination with other compositions, agents, drugs, biologics, etc. Accordingly, rAAV vectors alone and with other compositions, agents, drugs, biologics (proteins) can be incorporated into pharmaceutical compositions. Such pharmaceutical compositions are useful for, among other things, administration and delivery to a subject in vivo or ex vivo.

In particular embodiments, pharmaceutical compositions also contain a pharmaceutically acceptable carrier or excipient. Such excipients include any pharmaceutical agent that does not itself induce an immune response harmful to the individual receiving the composition, and which may be administered without undue toxicity.

As used herein the term “pharmaceutically acceptable” and “physiologically acceptable” mean a biologically acceptable formulation, gaseous, liquid or solid, or mixture thereof, which is suitable for one or more routes of administration, in vivo delivery or contact. A “pharmaceutically acceptable” or “physiologically acceptable” composition is a material that is not biologically or otherwise undesirable, e.g., the material may be administered to a subject without causing substantial undesirable biological effects. Thus, such a pharmaceutical composition may be used, for example in administering a nucleic acid, vector, viral particle or protein to a subject.

Pharmaceutically acceptable excipients include, but are not limited to, liquids such as water, saline, glycerol, sugars and ethanol. Pharmaceutically acceptable salts can also be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles.

The pharmaceutical composition may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding, free base forms. In other cases, a preparation may be a lyophilized powder which may contain any or all of the following: 1-50 mM histidine, 0.1%-2% sucrose, and 2-7% mannitol, at a pH range of 4.5 to 5.5, that is combined with buffer prior to use.

Pharmaceutical compositions include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. Such pharmaceutically acceptable carriers include tablets (coated or uncoated), capsules (hard or soft), microbeads, powder, granules and crystals. Supplementary active compounds (e.g., preservatives, antibacterial, antiviral and antifungal agents) can also be incorporated into the compositions.

Pharmaceutical compositions can be formulated to be compatible with a particular route of administration or delivery, as set forth herein or known to one of skill in the art. Thus, pharmaceutical compositions include carriers, diluents, or excipients suitable for administration by various routes.

Compositions suitable for parenteral administration comprise aqueous and non-aqueous solutions, suspensions or emulsions of the active compound, which preparations are typically sterile and can be isotonic with the blood of the intended recipient. Non-limiting illustrative examples include water, buffered saline, Hanks' solution, Ringer's solution, dextrose, fructose, ethanol, animal, vegetable or synthetic oils. Aqueous injection suspensions may contain substances which increase the viscosity of the suspension, such as sodium carboxymethyl cellulose, sorbitol, or dextran.

Additionally, suspensions of the active compounds may be prepared as appropriate oil injection suspensions. Suitable lipophilic solvents or vehicles include fatty oils such as sesame oil, or synthetic fatty acid esters, such as ethyl oleate or triglycerides, or liposomes. Optionally, the suspension may also contain suitable stabilizers or agents which increase the solubility of the compounds to allow for the preparation of highly concentrated solutions.

Cosolvents and adjuvants may be added to the formulation. Non-limiting examples of cosolvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters. Adjuvants include, for example, surfactants such as, soya lecithin and oleic acid; sorbitan esters such as sorbitan trioleate; and polyvinylpyrrolidone.

After pharmaceutical compositions have been prepared, they may be placed in an appropriate container and labeled for treatment. Such labeling could include amount, frequency, and method of administration.

Pharmaceutical compositions and delivery systems appropriate for the compositions, methods and uses of the invention are known in the art (see, e.g., Remington: The Science and Practice of Pharmacy (2003) 20^{th} ed., Mack Publishing Co., Easton, Pa.; Remington's Pharmaceutical Sciences (1990) 18^{th} ed., Mack Publishing Co., Easton, Pa.; The Merck Index (1996) 12^{th} ed., Merck Publishing Group, Whitehouse, N.J.; Pharmaceutical Principles of Solid Dosage Forms (1993), Technonic Publishing Co., Inc., Lancaster, Pa.; Ansel and Stoklosa, Pharmaceutical Calculations (2001) 11^{th} ed., Lippincott Williams & Wilkins, Baltimore, Md.; and Poznansky et al., Drug Delivery Systems (1980), R. L. Juliano, ed., Oxford, N.Y., pp. 253-315).

An “effective amount” or “sufficient amount” refers to an amount that provides, in single or multiple doses, alone or in combination, with one or more other compositions (therapeutic or immunosupprosive agents such as a drug), treatments, protocols, or therapeutic regimens agents, a detectable response of any duration of time (long or short term), an expected or desired outcome in or a benefit to a subject of any measurable or detectable degree or for any duration of time (e.g., for minutes, hours, days, months, years, or cured).

Doses can vary and depend upon the type, onset, progression, severity, frequency, duration, or probability of the disease to which treatment is directed, the clinical endpoint desired, previous or simultaneous treatments, the general health, age, gender, race or immunological competency of the subject and other factors that will be appreciated by the skilled artisan. The dose amount, number, frequency or duration may be proportionally increased or reduced, as indicated by any adverse side effects, complications or other risk factors of the treatment or therapy and the status of the subject. The skilled artisan will appreciate the factors that may influence the dosage and timing required to provide an amount sufficient for providing a therapeutic or prophylactic benefit.

The dose to achieve a therapeutic effect, e.g., the dose in vector genomes/per kilogram of body weight (vg/kg), will vary based on several factors including, but not limited to: route of administration, the level of heterologous polynucleotide expression required to achieve a therapeutic effect, the specific disease treated, any host immune response to the viral vector, a host immune response to the heterologous polynucleotide or expression product (protein), and the stability of the protein expressed. One skilled in the art can determine a rAAV/vector genome dose range to treat a patient having a particular disease or disorder based on the aforementioned factors, as well as other factors.

Generally, doses will range from at least 1×10⁸ vector genomes per kilogram (vg/kg) of the weight of the subject, or more, for example, 1×10⁹, 1×10¹⁰, 1×10¹¹, 1×10¹², 1×10¹³ or 1×10¹⁴, or more, vector genomes per kilogram (vg/kg) of the weight of the subject, to achieve a therapeutic effect. A rAAV dose in the range of 1×10¹⁰-1×10¹¹ vg/kg in mice, and 1×10¹²-1×10¹³ vg/kg in dogs have been effective. Doses can be less, for example, a dose of less than 6×10¹² vg/kg. More particularly, a dose of 5×10¹¹ vg/kg or 1×10¹² vg/kg.

rAAV vector doses can be at a level, typically at the lower end of the dose spectrum, such that there is not a substantial immune response against the heterologous nucleic acid sequence, the encoded protein or inhibitory nucleic acid, or rAAV vector. More particularly, a dose of up to but less than 6×10¹² vg/kg, such as about 5×10¹¹ to about 5×10¹² vg/kg, or more particularly, about 5×10¹¹ vg/kg or about 1×10¹² vg/kg.

The doses of an “effective amount” or “sufficient amount” for treatment (e.g., to ameliorate or to provide a therapeutic benefit or improvement) typically are effective to provide a response to one, multiple or all adverse symptoms, consequences or complications of the disease, one or more adverse symptoms, disorders, illnesses, pathologies, or complications, for example, caused by or associated with the disease, to a measurable extent, although decreasing, reducing, inhibiting, suppressing, limiting or controlling progression or worsening of the disease is a satisfactory outcome.

An effective amount or a sufficient amount can but need not be provided in a single administration, may require multiple administrations, and, can but need not be, administered alone or in combination with another composition (e.g., agent), treatment, protocol or therapeutic regimen. For example, the amount may be proportionally increased as indicated by the need of the subject, type, status and severity of the disease treated or side effects (if any) of treatment.

In addition, an effective amount or a sufficient amount need not be effective or sufficient if given in single or multiple doses without a second composition (e.g., another drug or agent), treatment, protocol or therapeutic regimen, since additional doses, amounts or duration above and beyond such doses, or additional compositions (e.g., drugs or agents), treatments, protocols or therapeutic regimens may be included in order to be considered effective or sufficient in a given subject. Amounts considered effective also include amounts that result in a reduction of the use of another treatment, therapeutic regimen or protocol, such as administration of recombinant enzyme (e.g., GAA) for treatment of an enzyme deficiency (e.g., Pompe disease) or administration of recombinant clotting factor protein (e.g., FVIII) for treatment of a clotting disorder (e.g., hemophilia A).

Accordingly, methods and uses of the invention also include, among other things, methods and uses that result in a reduced need or use of another compound, agent, drug, therapeutic regimen, treatment protocol, process, or remedy. Thus, in accordance with the invention, methods and uses of reducing need or use of another treatment or therapy are provided.

An effective amount or a sufficient amount need not be effective in each and every subject treated, nor a majority of treated subjects in a given group or population. An effective amount or a sufficient amount means effectiveness or sufficiency in a particular subject, not a group or the general population. As is typical for such methods, some subjects will exhibit a greater response, or less or no response to a given treatment method or use.

The term “ameliorate” means a detectable or measurable improvement in a subject's disease or symptom thereof, or an underlying cellular response. A detectable or measurable improvement includes a subjective or objective decrease, reduction, inhibition, suppression, limit or control in the occurrence, frequency, severity, progression, or duration of the disease, or complication caused by or associated with the disease, or an improvement in a symptom or an underlying cause or a consequence of the disease, or a reversal of the disease.

For Pompe disease, an effective amount would be an amount of GAA that inhibits or reduces glycogen production or accumulation, enhances or increases glycogen degradation or removal, or improves muscle tone and/or muscle strength in a subject, for example. For hemophilia A, an effective amount would be an amount that reduces frequency or severity of acute bleeding episodes in a subject, for example, or an amount that reduces clotting time as measured by a clotting assay, for example.

Therapeutic doses will depend on, among other factors, the age and general condition of the subject, the severity of the disease or disorder. A therapeutically effective amount in humans will fall in a relatively broad range that may be determined by a medical practitioner based on the response of an individual patient.

Compositions such as pharmaceutical compositions may be delivered to a subject, so as to allow production of the encoded protein or inhibitory nucleic acid. In a particular embodiment, pharmaceutical compositions comprise sufficient genetic material to enable a recipient to produce a therapeutically effective amount of a protein or inhibitory nucleic acid in the subject.

The compositions may be administered alone. In certain embodiments, a recombinant AAV particle provides a therapeutic effect without an immunosuppressive agent. The therapeutic effect optionally is sustained for a period of time, e.g., 2-4, 4-6, 6-8, 8-10, 10-14, 14-20, 20-25, 25-30, or 30-50 days or more, for example, 50-75, 75-100, 100-150, 150-200 days or more without administering an immunosuppressive agent. Accordingly, a therapeutic effect is provided for a period of time.

The compositions may be administered in combination with at least one other agent. In certain embodiments, rAAV vector is administered in conjunction with one or more immunosuppressive agents prior to, substantially at the same time or after administering a rAAV vector. In certain embodiments, e.g., 1-12, 12-24 or 24-48 hours, or 2-4, 4-6, 6-8, 8-10, 10-14, 14-20, 20-25, 25-30, 30-50, or more than 50 days following administering rAAV vector. Such administration of immunosuppressive agents after a period of time following administering rAAV vector if there is a decrease in the encoded protein or inhibitory nucleic acid after the initial expression levels for a period of time, e.g., 20-25, 25-30, 30-50, 50-75, 75-100, 100-150, 150-200 or more than 200 days following rAAV vector.

In certain embodiments, an immunosuppressive agent is an anti-inflammatory agent. In certain embodiments, an immunosuppressive agent is a steroid. In certain embodiments, an immunosuppressive agent is cyclosporine (e.g., cyclosporine A), mycophenolate, Rituximab or a derivative thereof. Additional particular agents include a stabilizing compound.

Compositions may be formulated and/or administered in any sterile, biocompatible pharmaceutical carrier, including, but not limited to, saline, buffered saline, dextrose, and water. The compositions may be formulated and/or administered to a patient alone, or in combination with other agents (e.g., co-factors) which influence hemostasis.

Methods and uses of the invention include delivery and administration systemically, regionally or locally, or by any route, for example, by injection or infusion. Delivery of the pharmaceutical compositions in vivo may generally be accomplished via injection using a conventional syringe, although other delivery methods such as convection-enhanced delivery are envisioned (See e.g., U.S. Pat. No. 5,720,720). For example, compositions may be delivered subcutaneously, epidermally, intradermally, intrathecally, intraorbitally, intramucosally, intraperitoneally, intravenously, intra-pleurally, intraarterially, orally, intrahepatically, via the portal vein, or intramuscularly. Other modes of administration include oral and pulmonary administration, suppositories, and transdermal applications. A clinician specializing in the treatment of patients with blood coagulation disorders may determine the optimal route for administration of the AAV vectors based on a number of criteria, including, but not limited to: the condition of the patient and the purpose of the treatment (e.g., enhanced or reduced blood coagulation).

Invention rAAV vectors, methods and uses can be combined with any compound, agent, drug, treatment or other therapeutic regimen or protocol having a desired therapeutic, beneficial, additive, synergistic or complementary activity or effect. Exemplary combination compositions and treatments include second actives, such as, biologics (proteins), agents (e.g., immunosuppressive agents) and drugs. Such biologics (proteins), agents, drugs, treatments and therapies can be administered or performed prior to, substantially contemporaneously with or following any other method or use of the invention.

The compound, agent, drug, treatment or other therapeutic regimen or protocol can be administered as a combination composition, or administered separately, such as concurrently or in series or sequentially (prior to or following) delivery or administration of a nucleic acid, vector, or rAAV particle. The invention therefore provides combinations in which a method or use of the invention is in a combination with any compound, agent, drug, therapeutic regimen, treatment protocol, process, remedy or composition, set forth herein or known to one of skill in the art. The compound, agent, drug, therapeutic regimen, treatment protocol, process, remedy or composition can be administered or performed prior to, substantially contemporaneously with or following administration of a nucleic acid, vector or rAAV particle of the invention, to a subject.

The invention is useful in animals including human and veterinary medical applications. Suitable subjects therefore include mammals, such as humans, as well as non-human mammals. The term “subject” refers to an animal, typically a mammal, such as humans, non-human primates (apes, gibbons, gorillas, chimpanzees, orangutans, macaques), a domestic animal (dogs and cats), a farm animal (poultry such as chickens and ducks, horses, cows, goats, sheep, pigs), and experimental animals (mouse, rat, rabbit, guinea pig). Human subjects include fetal, neonatal, infant, juvenile and adult subjects. Subjects include animal disease models, for example, mouse and other animal models of protein/enzyme deficiencies such as Pompe disease, blood clotting diseases such as HemA and others known to those of skill in the art.

Subjects appropriate for treatment in accordance with the invention include those having or at risk of producing an insufficient amount or having a deficiency in a functional gene product or produce an aberrant, partially functional or non-functional gene product, which can lead to disease. Subjects appropriate for treatment in accordance with the invention also include those having or at risk of producing an aberrant, or defective (mutant) gene product (protein) that leads to a disease such that reducing amounts, expression or function of the aberrant, or defective (mutant) gene product (protein) would lead to treatment of the disease, or reduce one or more symptoms or ameliorate the disease.

Subjects can be tested for an immune response, e.g., antibodies against AAV. Candidate subjects can therefore be screened prior to treatment according to a method of the invention. Subjects also can be tested for antibodies against AAV after treatment, and optionally monitored for a period of time after treatment. Subjects developing AAV antibodies can be treated with an immunosuppressive agent, or can be administered one or more additional amounts of AAV vector.

Subjects appropriate for treatment in accordance with the invention also include those having or at risk of producing antibodies against AAV. rAAV vectors can be administered or delivered to such subjects using several techniques. For example, AAV empty capsid (i.e., AAV lacking a heterologous nucleic acid) can be delivered to bind to the AAV antibodies in the subject thereby allowing the rAAV vector comprising the heterologous nucleic acid to transduce cells of the subject.

Ratio of AAV empty capsids to the rAAV vector can be between about 2:1 to about 50:1, or between about 2:1 to about 25:1, or between about 2:1 to about 20:1, or between about 2:1 to about 15:1, or between about 2:1 to about 10:1. Ratios can also be about 2:1, 3:1, 4:1, 5:1, 6:1, 7:1, 8:1, 9:1, or 10:1.

Amounts of AAV empty capsids to administer can be calibrated based upon the amount (titer) of AAV antibodies produced in a particular subject. AAV empty capsids can be of any serotype, for example, SEQ ID NO:1, SEQ ID NO:2, AAV1, AAV2, AAV3, AAV4, AAV5, AAV6, AAV7, AAV8, AAV9, AAV10, AAV11, AAV12, Rh10, Rh74 or AAV-2i8.

Alternatively or in addition to, rAAV vector can be delivered by direct intramuscular injection (e.g., one or more slow-twitch fibers of a muscle). In another alternative, a catheter introduced into the femoral artery can be used to delivery rAAV vectors to liver via the hepatic artery. Non-surgical means can also be employed, such as endoscopic retrograde cholangiopancreatography (ERCP), to deliver rAAV vectors directly to the liver, thereby bypassing the bloodstream and AAV antibodies. Other ductal systems, such as the ducts of the submandibular gland, can also be used as portals for delivering rAAV vectors into a subject that develops or has preexisting anti-AAV antibodies.

Administration or in vivo delivery to a subject can be performed prior to development of an adverse symptom, condition, complication, etc. caused by or associated with the disease. For example, a screen (e.g., genetic) can be used to identify such subjects as candidates for invention compositions, methods and uses. Such subjects therefore include those screened positive for an insufficient amount or a deficiency in a functional gene product, or that produce an aberrant, partially functional or non-functional gene product.

Administration or in vivo delivery to a subject in accordance with the methods and uses of the invention as disclosed herein can be practiced within 1-2, 2-4, 4-12, 12-24 or 24-72 hours after a subject has been identified as having the disease targeted for treatment, has one or more symptoms of the disease, or has been screened and is identified as positive as set forth herein even though the subject does not have one or more symptoms of the disease. Of course, methods and uses of the invention can be practiced 1-7, 7-14, 14-24, 24-48, 48-64 or more days, months or years after a subject has been identified as having the disease targeted for treatment, has one or more symptoms of the disease, or has been screened and is identified as positive as set forth herein.

A “unit dosage form” as used herein refers to physically discrete units suited as unitary dosages for the subject to be treated; each unit containing a predetermined quantity optionally in association with a pharmaceutical carrier (excipient, diluent, vehicle or filling agent) which, when administered in one or more doses, is calculated to produce a desired effect (e.g., prophylactic or therapeutic effect). Unit dosage forms may be within, for example, ampules and vials, which may include a liquid composition, or a composition in a freeze-dried or lyophilized state; a sterile liquid carrier, for example, can be added prior to administration or delivery in vivo. Individual unit dosage forms can be included in multi-dose kits or containers. rAAV particles, and pharmaceutical compositions thereof can be packaged in single or multiple unit dosage form for ease of administration and uniformity of dosage.

Subjects can be tested for protein activity to determine if such subjects are appropriate for treatment according to a method of the invention. Subjects also can be tested for amounts of protein according to a method of the invention. Such treated subjects can be monitored after treatment periodically, e.g., every 1-4 weeks, 1-6 months, 6-12 months, or 1, 2, 3, 4, 5 or more years.

Subjects can be tested for one or more liver enzymes for an adverse response or to determine if such subjects are appropriate for treatment according to a method of the invention. Candidate subjects can therefore be screened for amounts of one or more liver enzymes prior to treatment according to a method of the invention. Subjects also can be tested for amounts of one or more liver enzymes after treatment according to a method of the invention. Such treated subjects can be monitored after treatment for elevated liver enzymes, periodically, e.g., every 1-4 weeks, 1-6 months, 6-12 months, or 1, 2, 3, 4, 5 or more years.

Exemplary liver enzymes include alanine aminotransferase (ALT), aspartate aminotransferase (AST), and lactate dehydrogenase (LDH), but other enzymes indicative of liver damage can also be monitored. A normal level of these enzymes in the circulation is typically defined as a range that has an upper level, above which the enzyme level is considered elevated, and therefore indicative of liver damage. A normal range depends in part on the standards used by the clinical laboratory conducting the assay.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

All patents, patent applications, publications, and other references, GenBank citations and ATCC citations cited herein are incorporated by reference in their entirety. In case of conflict, the specification, including definitions, will control.

Various terms relating to the biological molecules of the invention are used hereinabove and also throughout the specification and claims.

All of the features disclosed herein may be combined in any combination. Each feature disclosed in the specification may be replaced by an alternative feature serving a same, equivalent, or similar purpose. Thus, unless expressly stated otherwise, disclosed features are an example of a genus of equivalent or similar features.

As used herein, the singular forms “a”, “and,” and “the” include plural referents unless the context clearly indicates otherwise. Thus, for example, reference to “a nucleic acid” includes a plurality of such nucleic acids, reference to “a vector” includes a plurality of such vectors, and reference to “a virus” or “particle” includes a plurality of such viruses/particles.

As used herein, all numerical values or numerical ranges include integers within such ranges and fractions of the values or the integers within ranges unless the context clearly indicates otherwise. Thus, to illustrate, reference to 80% or more identity, includes 81%, 82%, 83%, 84%, 85%, 86%, 87%, 88%, 89%, 90%, 91%, 92%, 93%, 94% etc., as well as 81.1%, 81.2%, 81.3%, 81.4%, 81.5%, etc., 82.1%, 82.2%, 82.3%, 82.4%, 82.5%, etc., and so forth.

Reference to an integer with more (greater) or less than includes any number greater or less than the reference number, respectively. Thus, for example, a reference to less than 100, includes 99, 98, 97, etc. all the way down to the number one (1); and less than 10, includes 9, 8, 7, etc. all the way down to the number one (1).

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth.

Reference to a series of ranges includes ranges which combine the values of the boundaries of different ranges within the series. Thus, to illustrate, reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-850, includes ranges of 1-20, 1-30, 1-40, 1-50, 1-60, 10-30, 10-40, 10-50, 10-60, 10-70, 10-80, 20-40, 20-50, 20-60, 20-70, 20-80, 20-90, 50-75, 50-100, 50-150, 50-200, 50-250, 100-200, 100-250, 100-300, 100-350, 100-400, 100-500, 150-250, 150-300, 150-350, 150-400, 150-450, 150-500, etc.

The invention is generally disclosed herein using affirmative language to describe the numerous embodiments and aspects. The invention also specifically includes embodiments in which particular subject matter is excluded, in full or in part, such as substances or materials, method steps and conditions, protocols, or procedures. For example, in certain embodiments or aspects of the invention, materials and/or method steps are excluded. Thus, even though the invention is generally not expressed herein in terms of what the invention does not include aspects that are not expressly excluded in the invention are nevertheless disclosed herein.

A number of embodiments of the invention have been described. Nevertheless, one skilled in the art, without departing from the spirit and scope of the invention, can make various changes and modifications of the invention to adapt it to various usages and conditions. Accordingly, the following examples are intended to illustrate but not limit the scope of the invention claimed in any way.

### EXAMPLES

The following are representative non-liming examples of affinity resins (GE Healthcare) that could be used to generate an AAV capsid specific antibody affinity matrix. AAV capsid proteins contain amino acids that may be cross linked to the various chromatography media described below. Comparable and/or suitable materials from other manufacturers of affinity resins to which AAV capsids could be attached may also be used or made accordingly.

### Example 1

### CNBr-Activated Sepharose 4 Fast Flow

CNBr-activated Sepharose 4 Fast Flow is a pre-activated chromatography medium for coupling of large amino-containing ligands.
CNBr-activated BioProcess medium designed for coupling of large amino-containing ligands.
Rapid and efficient coupling
This resin allows for multi-point attachment of protein ligands which minimizes ligand leakage
BioProcess medium supported for industrial applications and well-established in approved processes

CNBr-activated Sepharose 4 Fast Flow is based on the established Sepharose Fast Flow platform. The resin is composed of cross-linked 4% agarose beads that have been pre-activated with cyanogen bromide. CNBr-activated Speharose 4 Fast Flow is designed for multipoint attachment of protein ligands containing amino groups.

The preparation and use of affinity chromatography media by coupling biospecific ligands to CNBr-activated Sepharose 4 Fast Flow is a widely used, and well-documented approach, with an easy, rapid and efficient coupling procedure.

CNBr-activated Sepharose 4 Fast Flow is available in a range of different bulk pack sizes and convenient pre-packed formats for easy scale-up and process development.

As member of the BioProcess media range, CNBr-activated Sepharose 4 Fast Flow meets industrial demands with security of supply and comprehensive technical and regulatory support.

### Example 2

### Activated Thiol Sepharose 4B

Activated Thiol Sepharose 4B medium is a medium used for reversible immobilization of molecules containing thiol groups under mild conditions. Optimized for immobilization of large molecules
Reversible coupling of proteins and large biomolecules with thiol groups to Sepharose 4B via a glutathione spacer arm
The ligand is a mixed disulphide formed between 2,2′-dipyridyl disulphide and glutathione coupled to CNBr-activated Sepharose 4B.
Well suited for covalent chromatography of large molecules such as enzymes and nucleic acids
Gel also reacts with heavy metal ions and with alkyl and aryl halides. Addition reactions occur with compounds containing C═O, C═C, and N═N bonds
Separate thiol-containing proteins from non-thiol-containing proteins

Activated Thiol Sepharose 4B is a mixed disulphide formed between 2,2′-dipyridyl disulphide and glutathione coupled to CNBr-activated Sepharose 4B. Activated Thiol Sepharose 4B reacts with solutes containing thiol groups under mild conditions to form mixed disulphides. This reaction forms the basis of covalent chromatography and a procedure for immobilizing thiol containing biomolecules.

### Example 3

### EAH Sepharose 4B

EAH Sepharose pre-activated media is used for coupling compounds containing carboxyl groups to Sepharose 4B through carbodiimide-based coupling via an 11-atom spacer arm.
Stable carbodiimide-based coupling of carboxyl groups to Sepharose 4B via an 11-atom hydrophilic spacer arm permits very stable coupling

### Example 4

### Epoxy-Activated Sepharose 6B

Epoxy-activated Sepharose 6B is a pre-activated medium for immobilization of various ligands including sugars through coupling of hydroxy, amino or thiol groups on the ligand to Sepharose 6B via a 12-atom hydrophilic spacer arm
Epoxy-activated Sepharose 6B can be used to couple sugars and other carbohydrates via stable ether linkages to hydroxyl groups.

Epoxy-activated Sepharose 6B is a pre-activated medium for immobilization of various ligands. Epoxy-activated Sepharose 6B can be used to couple sugars and other carbohydrates via stable ether linkages to hydroxyl groups. Other ligands can be coupled through hydroxyl, amino or thiol groups. The medium has a long hydrophilic spacer arm which makes it particular suitable for immobilization of small molecules. Epoxy-activated Sepharose 6B is formed by reacting Sepharose 6B with 1,4-bis (2,3-epoxy-propoxy-) butane.

### Example 5

### Purified AAV Capsid, May be Essentially GMP Grade

A typical feature of the AAV capsid protein to be used in combination with an appropriate resin or matrix to generate an AAV capsid affinity matrix is purity. Generation of AAV in cell culture is complex, and its separation from the abundant non AAV components (impurities and contaminants) co-generated is important. Specifically, AAV capsid particles are purified from production cell and cell culture media derived impurities. The presence of high impurity levels in AAV capsid preparation used to generated affinity matrices will result in lower efficiency and lower binding specificity when used in the context of apheresis. One exemplary purification of AAV particles is under current Good Manufacturing Practices (GMP) for human parenteral products because the envisaged affinity matrix will be in contact with human subject blood product in the plasmapheresis process.

### Example 6

### AAV Capsid Material for Production of the Affinity Matrix

One typical form of capsid material for use in generating an AAV capsid affinity matrix for plasmapheresis is AAV “empty” capsids, which are AAV particles that lack a transgene. Affinity matrix-bound AAV empty capsid would be predicted to display the same surface epitopes as the corresponding AAV vector.

For example, highly purified empty capsid derived from AAV capsid variant SEQ ID NO:1 or 2 could be coupled to CNBr-activated Sepharose resin. In a human subject with severe hemophilia A and with an AAV-SEQ ID NO:1 or 2 antibody titer of 1:100, sufficient plasmapheresis as set forth herein with AAV binding antibody affinity matrix prepared using highly purified AAV-SEQ ID NO:1 or 2 empty capsids is predicted to reduce the subject's AAV-SEQ ID NO:1 or 2 antibody titer, for example, to 1:1. Less than about twelve hours or typically less than about six hours of completion of the plasmapheresis protocol, this subject would then be treated with AAV-AAV-SEQ ID NO:1 or 2 expressing a human coagulation Factor VIII, achieving efficient gene transfer and subsequently expressing therapeutic levels of circulating FVIII. At higher initial AAV titers (greater than 1:100), treatment after plasmapheresis protocol occurs sooner.

Alternatively, empty capsids prepared for any other known AAV capsid serotype or capsid variant could similarly be used to reduce antibody titers to that specific AAV capsid serotype or capsid variant, thereby enabling efficient gene transfer with a corresponding AAV vector expressing any therapeutic transgene.

Still further in another alternative, any of AAV VP1, VP2 and VP3 capsid proteins from any naturally occurring AAV capsid serotype or synthetic AAV capsid variant, used alone or in combination in any stoichiometry, could similarly be used to reduce AAV antibody titers to enable efficient gene transfer with a corresponding viral vector expressing any therapeutic gene.

### Example 7

### Other Viral Vectors Materials for Production of the Affinity Matrix

Alternatively, other viral vector or viral vector protein, from viruses that can be used to achieve gene transfer may be used to develop an antibody affinity matrix and implement the methods as set forth herein. Exemplary, viruses are from the following virus families: Picornaviridae, Caliciviridae, Astroviridae, Togaviridae, Flaviviridae, Coronoviridae, Rhabdoviridae, Filoviridae, Paromyxoviridae, Orthomyxoviridae, Bunyaviridae, Arenaviridae, Reoviridae, Etroviridae, Papoviridae, Adenoviridae, Parvoviridae, Herpesviridae, Poxviridae, Hepadnaviridae, could be used to reduce the respective virus antibody titers to enable efficient gene transfer with a corresponding vector expressing any therapeutic gene.

### Example 8

Re Administration and/or Serial Administration of Vector for Therapeutic Benefit

In addition to the inhibition of therapeutic gene transfer by AAV vectors by pre-existing antibodies arising naturally in the human population, capsid specific antibodies are may increase following administration of AAV vectors (and other vectors as set forth herein) themselves. The use of AAV capsid affinity plasmapheresis can similarly reduce AAV capsid antibodies caused by a prior administration of a gene therapy vector, enabling efficient gene transfer upon re-administration. This process could be performed in a serial manner over an extended period of time to gradually increase the level of therapeutic gene expression in human subjects. For example, it is believed that blood level of coagulation factor FVIII following AAV based gene transfer in a young child with severe hemophilia A will gradually decrease as the child grows. Periodic re administration during childhood and adolescence using the AAV capsid affinity plasmapheresis protocol disclosed herein will enable maintenance of therapeutic levels of FVIII as the child grows and through adulthood.

### Example 9

### Antibody Rebound Rate Calculation

IgG half life (human)=20 days

IgG concentration in human serum ranges from 8-18 mg/mL, will use 12 mg/mLm

Expo decay formula HID=No (1/2)t/t 1/2 where tin days

Demonstration of formula: H(0)=12 (1/2)0/20=12(1)=12 mg/mL
H(20)=12 (1/2)20/20=12(1/2)=6 mg/mL

$$N = IgG loss \begin{matrix}1 d = 24 h N ( t = 1 ) = 12 {( 1 \text{/} 2 )}^{1 / 20} = 12 {( 0.5 )}^{0.05} = 11.591 mg \text{/} mL \\ 0.5 d = 12 h N ( 0.5 ) = 12 {( 1 \text{/} 2 )}^{0.5 / 20} = 12 {( 0.5 )}^{0.025} = 11.794 mg \text{/} mL \\ 0.25 d = 6 h N ( 0.25 ) = 12 {( 1 \text{/} 2 )}^{0.25 / 20} = 12 {( 0.5 )}^{0.0125} = 11.896 mg \text{/} mL \\ = 3 h H ( 0.125 ) = 12 {( 1 \text{/} 2 )}^{0.125 / 20} = 12 {( 0.5 )}^{0.00625} = 11.948 mg \text{/} mL \\ = 1 h N ( 0.042 ) = 12 ( 1 \text{/} 2 ) ⋀ 0.042 \text{/} 20 = 12 ( 0.5 ) ⋀ 0.00208 \\ = 11.983 mg \text{/} mL\end{matrix} steady state 12 mg \text{/} mL means an equal synthesis rate$$

IgG Synthesis

| | | |
|---|---|---|
| 24 | h | 12 − 11.591 = 0.409 mg/m² ÷ 12 = 3.41% |
| 12 | h | 12 − 11.794 = 0.206 mg/m² ÷ 12 = 1.72% |
| 6 | h | 12 − 11.896 = 0.104 mg/m² ÷ 12 = 0.87% |
| 3 | h | 12 − 11.948 = 0.052 mg/m² ÷ 12 = 0.43% |
| 1 | h | 12 − 11.983 = 0.017 mg/mL/12 = 0.15% |

Assume synthesis rate distributes comparably across all IgGs

[symbol]

an AAV capsid IgG starting at 1:100 reduced to 1:1 by capsid plasmapheresis rebounds to

| | |
|---|---|
| 1:4.4 | at 24 hours |
| 1:.7 | at 12 hours |
| 1:1.9 | at 6 hours |
| 1:1.43 | at 3 hours |
| 1:1.15 | at 1 hour |

Table 1 shows a broader range of titer rebound rates as a function of initial AAV capsid IgG titers (namely, 1:10. 1:230, 1:100 as above, 1:300, 1:1000, 1:3000, 1:10000). In particular, Table 1 shows that subjects having an AAV antibody titer up to 1:1000 can be administered an AAV vector for gene therapy within about 1 hour after plasmapheresis; subjects having an AAV antibody titer up to 1:300 can be administered an AAV vector for gene therapy within about 3 hours after plasmapheresis; subjects having an AAV antibody titer up to 1:100 can be administered an AAV vector for gene therapy within about 6 hours after plasmapheresis; subjects having an AAV antibody titer up to 1:100 can also be administered an AAV vector for gene therapy within about 12 hours after plasmapheresis; and subjects having an AAV antibody titer up to 1:30 can be administered an AAV vector for gene therapy within about 24 hours after plasmapheresis.

TABLE 1

| | Time post plasmapheresis (hours) | | | | | | |
|---|---|---|---|---|---|---|---|
| | Pre | 0 | 1 | 3 | 6 | 12 | 24 |
| AAV | 1:10 | s1:1 | 1:1.02 | 1:1.04 | 1:1.09 | 1:1.17 | 1:1.34 |
| Antibody | 1:30 | s1:1 | 1:1.04 | 1:1.13 | 1:1.26 | 1:1.52 | 1:2.02 |
| titer | 1:100 | s1:1 | 1:1.15 | 1:1.43 | 1:1.87 | 1:2.72 | 1:4.41 |
| | 1:300 | s1:1 | 1:1.44 | 1:2.29 | 1:3.6 | 1:6.2 | 1:23.2 |
| | 1:1000 | s1:1 | 1:2.45 | 1:5.3 | 1:9.7 | 1:18.2 | 1:35 |
| | 1:3000 | s1:1 | 1:5.4 | 1:13.9 | 1:27 | 1:53 | |
| | 1:10000 | s1:1 | 1:15.5 | 1:64 | | | |

### Example 9

### Representative AAV Capsid (VP1) Proteins

| AAV-SPK VP1 Capsid (SEQ ID NO:1) | |
|---|---|
| 1 | MAADGYLPDWLEDNLSEGIREWWDLKPGAPKPKANQQKQDNGRGLVLPGYKYLGPFNGLD |
| 61 | KGEPVNAADAAALEHDKAYDQQLQAGDNPYLRYNHADAEFQERLQEDTSFGGNLGRAVFQ |
| 121 | AKKRVLEPLGLVESPVKTAPGKKRPVEPSPQRSPDSSTGIGKKGQQPAKKRLNFGQTGDS |
| 181 | ESVPDPQPIGEPPAAPSGVGPNTMAAGGGAPMADNNEGADGVGSSSGNWHCDSTWLGDRV |
| 241 | ITTSTRTWALPTYNNHLYKQISNGTSGGSTNDNTYFGYSTPWGYFDFNRFHCHFSPRDWQ |
| 301 | RLINNNWGFRPKRLNFKLFNIQVKEVTQNEGTKTIANNLTSTIQVFTDSEYQLPYVLGSA |
| 361 | HQGCLPPFPADVFMIPQYGYLTLNNGSQAVGRSSFYCLEYFPSQMLRTGNNFEFSYNFED |
| 421 | VPFHSSYAHSQSLDRLMNPLIDQYLYYLSRTQSTGGTAGTQQLLFSQAGPNNMSAQAKNW |
| 481 | LPGPCYRQQRVSTTLSQNNNSNFAWTGATKYHLNGRDSLVNPGVAMATHKDDEERFFPSS |
| 541 | GVLMFGKQGAGKDNVDYSSVMLTSEEEIKTTNPVATEQYGVVADNLQQQNAAPIVGAVNS |
| 601 | QGALPGMVWQNRDVYLQGPIWAKIPHTDGNFHPSPLMGGFGLKHPPPQILIKNTPVPADP |
| 661 | PTTFNQAKLASFITQYSTGQVSVEIEWELQKENSKRWNPEIQYTSNYYKSTNVDFAVNTE |
| 721 | GTYSEPRPIGTRYLTRNL |

AAV-LKO3 VP1 Capsid (SEQ ID NO:2)
MAADGYLPDWLEDNLSEGIREWWALQPGAPKPKANQQHQDNARGLVLPGYKYLGPGNGLDKGEPVNAADA
AALEHDKAYDQQLKAGDNPYLKYNHADAEFQERLKEDTSFGGNLGRAVFQAKKRLLEPLGLVEEAAKTAP
GKKRPVDQSPQEPDSSSGVGKSGKQPARKRLNFGQTGDSESVPDPQPLGEPPAAPTSLGSNTMASGGGAP
MADNNEGADGVGNSSGNWHCDSQWLGDRVTTSTRTWALPTYNNHLYKQISSQSGASNDNHYFGYSTPWG
YFDFNRFHCHFSPRDWQRLINNNWGFRPKKLSFKLFNIQVKEVTQNDGTTTIANNLTSTVQVFTDSEYQL
PYVLGSAHQGCLPPFPADVFMVPQYGYLTLNNGSQAVGRSSFYCLEYFPSQMLRTGNNFQFSYTFEDVPF
HSSYAHSQSLDRLMNPLIDQYLYYLNRTQGTTSGTTNQSRLLFSQAGPQSMSLQARNWLPGPCYRQQRLS
KTANDNNNSNFPWTAASKYHLNGRDSLVNPGPAMASHKDDEEKFFPMHGNLIFGKEGTTASNAELDNVMI
TDEEEIRTTNPVATEQYGTVANNLQSSNTAPTTRTVNDQGALPGMVWQDRDVYLQGPIWAKIPHTDGHFH
PSPLMGGFGLKHPPPQIMIKNTPVPANPPTTFSPAKFASFITQYSTGQVSVEIEWELQKENSKRWNPEIQ
NLYTSNYNKSVNVDFTVDTNGVYSEPRPIGTRYLTRPL

What is claimed is:

1. A method of treating a subject in need of treatment for a disease caused by a loss of function or activity of a protein comprising: (a) removing, reducing, depleting, inhibiting, inactivating or capturing AAV binding antibodies from a blood product obtained from the subject by a process comprising apheresis; and (b) administering an amount of a recombinant adeno-associated virus (rAAV) vector comprising a heterologous polynucleotide that encodes a protein or peptide that provides or supplements a function or activity of the protein.

2. A method of treating a subject in need of treatment for a disease caused by a gain of function, activity or expression, of a protein comprising: (a) removing, reducing, depleting, inhibiting, inactivating or capturing AAV binding antibodies from a blood product obtained from the subject by a process comprising apheresis; and (b) administering an amount of a recombinant adeno-associated virus (rAAV) vector comprising a heterologous polynucleotide that is transcribed into a nucleic acid that inhibits, decreases or reduces expression of the gain of function, activity or expression of the protein.

3. The method of claim 1 or 2, wherein the apheresis process comprises an AAV binding antibody affinity matrix attached to or immobilized on a substrate.

4. The method of claim 3, wherein the AAV binding antibody affinity matrix comprises an AAV capsid or AAV capsid fragment attached to or immobilized on a substrate that binds to the AAV binding antibodies in the blood product.

5. The method of any of claims 3-5, wherein the AAV binding antibody affinity matrix immobilized on a substrate is disposed within a column, apparatus, chamber, device, filter, cartridge, tube having an inlet and an outlet for extracorporeal or intracorporeal removal or depletion of AAV binding antibodies from the blood product upon contact with the AAV binding antibody affinity matrix.

6. The method of any one of claims 3-5, wherein the AAV binding antibody affinity matrix comprises naturally occurring or a non-natural or synthetic intact AAV empty capsids.

7. The method of any one of claims 3-5, wherein the AAV binding antibody affinity matrix comprises an AAV VP1, VP2 and/or VP3 capsid protein or fragment thereof.

8. The method of any one of claims 3-7, wherein the AAV binding antibody affinity matrix comprises a naturally occurring or a non-natural or synthetic AAV VP1, VP2 and/or VP3 capsid protein.

9. The method of any one of claims 3-8, wherein the AAV binding antibody affinity matrix comprises a naturally occurring or a non-natural or synthetic AAV VP1, VP2 and/or VP3 capsid protein monomer.

10. The method of any one of claims 3-9, wherein the AAV binding antibody affinity matrix comprises a naturally occurring or a non-natural or synthetic AAV VP1, VP2 and/or VP3 capsid protein polymer.

11. The method of any one of claims 3-10, wherein the AAV binding antibody affinity matrix comprises AAV VP1, VP2 and/or VP3 capsid protein having 60% or more sequence identity to a naturally occurring or a non-natural or synthetic AAV capsid protein.

12. The method of any one of claims 3-11, wherein the AAV binding antibody affinity matrix comprises AAV VP1, VP2 and/or VP3 capsid protein having 60% or more sequence identity to an AAV VP1, VP2 and/or VP3 capsid protein selected from the group consisting of AAV1, AAV2, AAV3, AAV4, AAV5, AAV6, AAV7, AAV8, AAV9, AAV10, Rh10, Rh74, SEQ ID NO:1 and SEQ ID NO:2 VP1, VP2 and/or VP3 capsid proteins.

13. The method of any one of claims 3-12, wherein the AAV binding antibody affinity matrix comprises AAV VP1, VP2 and/or VP3 capsid protein having 60% or more sequence identity to SEQ ID NO:1 or SEQ ID NO:2.

14. The method of any one of claims 3-5, wherein the AAV binding antibody affinity matrix comprises an anti-idiotype antibody that binds to the AAV binding antibodies in the blood product.

15. The method of claim 14, wherein the anti-idiotype antibody that binds to AAV binding antibodies binds to AAV1, AAV2, AAV3, AAV4, AAV5, AAV6, AAV7, AAV8, AAV9, AAV10, Rh10, Rh74, SEQ ID NO:1 and/or SEQ ID NO:2 capsid protein(s), or a derivative or amino acid substitution of AAV1, AAV2, AAV3, AAV4, AAV5, AAV6, AAV7, AAV8, AAV9, AAV10, Rh10, Rh74, SEQ ID NO:1 and/or SEQ ID NO:2 capsid protein(s).

16. The method of claim 14 or 15, wherein the anti-idiotype antibody that binds to AAV binding antibodies is an antibody fragment.

17. The method of any one of claims 1-16, wherein the anti-idiotype antibody that binds to AAV binding antibodies is an IgG, IgA, IgM, IgE or IgD.

18. The method of any one of claims 14-16, wherein the AAV binding antibody affinity matrix is GMP grade.

19. The method of any one of claims 1-18, wherein leaching of the AAV binding antibody affinity matrix into the blood product obtained from the subject, if the blood product is reintroduced into the subject, does not substantially harm the subject.

20. The method of any one of claims 1-19, wherein the AAV binding antibody comprises an IgG, IgM, IgA or IgD that binds to AAV capsid protein.

21. The method of any one of claims 1-20, wherein the substrate and/or column, apparatus, chamber, device, filter, cartridge, or tube is configured from plastic or glass.

22. The method of any one of claims 1-21, wherein the AAV binding antibody affinity matrix, substrate and/or column, apparatus, chamber, device, filter, cartridge, tube are sterile.

23. The method of any one of claims 1-22, wherein the AAV binding antibodies present in the blood product prior to the apheresis process are more than about 1:100, where 1 part of the blood product diluted in 100 parts of isotonic buffer results in 50% AAV neutralization.

24. The method of any one of claims 1-23, wherein the AAV binding antibodies present in the blood product prior to the apheresis process are more than about 1:1000, where 1 part of the blood product diluted in 1000 parts of isotonic buffer results in 50% AAV neutralization.

25. The method of any one of claims 1-24, wherein 20-50%, 50-75%, 75-90%, 90-95% or 95% or more of the AAV binding antibodies present in the blood product are removed.

26. The method of any one of claims 1-25, wherein the AAV binding antibodies present in the blood product after the apheresis process is less than about 1:10, where 1 part of the blood product diluted in 10 parts of isotonic buffer results in 50% AAV neutralization.

27. The method of any one of claims 1-26, wherein the AAV binding antibodies present in the blood product after the apheresis process is less than about 1:5, where 1 part of the blood product diluted in 5 parts of isotonic buffer results in 50% AAV neutralization.

28. The method of any one of claims 1-27, wherein the ratio of AAV binding antibodies present in the blood product after the apheresis process is less than about 1:4, where 1 part of the blood product diluted in 4 parts of isotonic buffer results in 50% AAV neutralization.

29. The method of any one of claims 1-27, wherein the ratio of AAV binding antibodies present in the blood product after the apheresis process is less than about 1:3, where 1 part of the blood product diluted in 3 parts of isotonic buffer results in 50% AAV neutralization.

30. The method of any one of claims 1-29, wherein the ratio of AAV binding antibodies present in the blood product after the apheresis process is less than about 1:2, where 1 part of the blood product diluted in 2 parts of isotonic buffer results in 50% AAV neutralization.

31. The method of any one of claims 1-30, wherein the ratio of AAV binding antibodies present in the blood product after the apheresis process is less than about 1:1, where 1 part of the blood product diluted in 1 parts of isotonic buffer results in 50% AAV neutralization.

32. The method of any one of claims 1-31, wherein all or a part of the blood product after the method is reintroduced or reinfused into the subject.

33. The method of any one of claims 1-32, wherein the subject, after step (b) receives a blood product from a donor.

34. The method of any one of claims 1-33, wherein step (b) is performed within about 72 hours after (a).

35. The method of any one of claims 1-33, wherein step (b) is performed within about 48 hours after (a).

36. The method of any one of claims 1-33, wherein step (b) is performed within about 1-48 hours after (a).

37. The method of any one of claims 1-33, wherein step (b) is performed within about 36 hours after (a).

38. The method of any one of claims 1-33, wherein step (b) is performed within about 24 hours after (a).

39. The method of any one of claims 1-33, wherein step (b) is performed within about 1-24 hours after (a).

40. The method of any one of claims 1-33, wherein step (b) is performed within about 12 hours after (a).

41. The method of any one of claims 1-33, wherein step (b) is performed within about 6 hours after (a).

42. The method of any one of claims 1-33, wherein step (b) is performed within about 3 hours after (a).

43. The method of any one of claims 1-33, wherein step (b) is performed within about 30 minutes to 6 hours after (a).

44. The method of any one of claims 1-33, wherein step (b) is performed within about 30 minutes to 3 hours after (a).

45. The method of any one of claims 1-44, further comprising after step (a) but before step (b) analyzing a sample from the subject for the amount of AAV binding antibodies present in the sample.

46. The method of any one of claims 1-45, further comprising after step (b) analyzing a sample from the subject for the amount of AAV binding antibodies present in the sample.

47. The method of claim 45 or 46, wherein the sample analyzed sample from the subject is a blood product.

48. The method of any one of claims 1-47, wherein the blood product is plasma.

49. The method of any of claims 1-48, wherein the subject has a lung disease (e.g., cystic fibrosis), a bleeding disorder (e.g., hemophilia A or hemophilia B with or without inhibitors), thalassemia, a blood disorder (e.g., anemia), Alzheimer's disease, Parkinson's disease, Huntington's disease, amyotrophic lateral sclerosis (ALS), epilepsy, lysosomal storage diseases, a copper or iron accumulation disorders (e.g., Wilson's or Menkes disease) lysosomal acid lipase deficiency, a neurological or neurodegenerative disorder, cancer, type 1 or type 2 diabetes, Gaucher's disease, Hurler's disease, adenosine deaminase deficiency, a metabolic defect (e.g., glycogen storage diseases), a retinal degenerative disease (such as RPE65 deficiency, choroideremia, and other diseases of the eye), a disease of solid organs (e.g., brain, liver, kidney, heart), or an infectious viral (e.g., hepatitis B and C, HIV, etc.), bacterial or fungal disease.

50. The method of any one of claims 1-49, wherein the disease is caused by lost or reduced expression of a gene that encodes the protein.

51. The method of any one of claims 1-50, wherein the disease is a blood clotting disorder.

52. The method of any one of claims 1-51, wherein the disease is hemophilia A, hemophilia A patients with inhibitory antibodies, hemophilia B, a deficiency in any coagulation Factor: VII, VIII, IX and X, XI, V, XII, II, von Willebrand factor, or a combined FV/FVIII deficiency, or thalassemia, vitamin K epoxide reductase C1 deficiency or gamma-carboxylase deficiency.

53. The method of any one of claims 1-50, wherein the disease is anemia, bleeding associated with trauma, injury, thrombosis, thrombocytopenia, stroke, coagulopathy, disseminated intravascular coagulation (DIC); over-anticoagulation associated with heparin, low molecular weight heparin, pentasaccharide, warfarin, small molecule antithrombotics (i.e. FXa inhibitors); and platelet disorders such as, Bernard Soulier syndrome, Glanzman thromblastemia, or storage pool deficiency.

54. The method of any one of claims 1-50, wherein the disease affects or originates in the central nervous system (CNS).

55. The method of any one of claims 1-50, wherein the disease is a neurodegenerative disease.

56. The method of claim 54 or 55, wherein the CNS or neurodegenerative disease is Alzheimer's disease, Huntington's disease, ALS, hereditary spastic hemiplegia, primary lateral sclerosis, spinal muscular atrophy, Kennedy's disease, a polyglutamine repeat disease, or Parkinson's disease.

57. The method of claim 55 or 56, wherein the CNS or neurodegenerative disease is a polyglutamine repeat disease.

58. The method of claim 57, wherein the polyglutamine repeat disease is a spinocerebellar ataxia (SCA1, SCA2, SCA3, SCA6, SCA7, or SCA17).

59. The method of any one of claim 1 or 3-58, wherein the heterologous polynucleotide encodes a protein selected from the group consisting of insulin, glucagon, growth hormone (GH), parathyroid hormone (PTH), growth hormone releasing factor (GRF), follicle stimulating hormone (FSH), luteinizing hormone (LH), human chorionic gonadotropin (hCG), vascular endothelial growth factor (VEGF), angiopoietins, angiostatin, granulocyte colony stimulating factor (GCSF), erythropoietin (EPO), connective tissue growth factor (CTGF), basic fibroblast growth factor (bFGF), acidic fibroblast growth factor (aFGF), epidermal growth factor (EGF), transforming growth factor α (TGFα), platelet-derived growth factor (PDGF), insulin growth factors I and II (IGF-I and IGF-II), TGFβ, activins, inhibins, bone morphogenic protein (BMP), nerve growth factor (NGF), brain-derived neurotrophic factor (BDNF), neurotrophins NT-3 and NT4/5, ciliary neurotrophic factor (CNTF), glial cell line derived neurotrophic factor (GDNF), neurturin, agrin, netrin-1 and netrin-2, hepatocyte growth factor (HGF), ephrins, noggin, sonic hedgehog and tyrosine hydroxylase.

60. The method of any one of claim 1 or 3-58, wherein the heterologous polynucleotide encodes a protein selected from the group consisting of thrombopoietin (TPO), interleukins (IL1 through IL-17), monocyte chemoattractant protein, leukemia inhibitory factor, granulocyte-macrophage colony stimulating factor, Fas ligand, tumor necrosis factors α and β, interferons α, β, and γ, stem cell factor, flk-2/flt3 ligand, IgG, IgM, IgA, IgD and IgE, chimeric immunoglobulins, humanized antibodies, single chain antibodies, T cell receptors, chimeric T cell receptors, single chain T cell receptors, class I and class II MHC molecules.

61. The method of any one of claim 1 or 3-58, wherein the heterologous polynucleotide encodes CFTR (cystic fibrosis transmembrane regulator protein), a blood coagulation (clotting) factor (Factor XIII, Factor IX, Factor VIII, Factor X, Factor VII, Factor VIIa, protein C, etc.) a gain of function blood coagulation factor, an antibody, retinal pigment epithelium-specific 65 kDa protein (RPE65), erythropoietin, LDL receptor, lipoprotein lipase, ornithine transcarbamylase, β-globin, α-globin, spectrin, α-antitrypsin, adenosine deaminase (ADA), a metal transporter (ATP7A or ATP7), sulfamidase, an enzyme involved in lysosomal storage disease (ARSA), hypoxanthine guanine phosphoribosyl transferase, β-25 glucocerebrosidase, sphingomyelinase, lysosomal hexosaminidase, branched-chain keto acid dehydrogenase, a hormone, a growth factor, insulin-like growth factor 1 or 2, platelet derived growth factor, epidermal growth factor, nerve growth factor, neurotrophic factor-3 and -4, brain-derived neurotrophic factor, glial derived growth factor, transforming growth factor α and β, a cytokine, α-interferon, β-interferon, interferon-γ, interleukin-2, interleukin-4, interleukin 12, granulocyte-macrophage colony stimulating factor, lymphotoxin, a suicide gene product, herpes simplex virus thymidine kinase, cytosine deaminase, diphtheria toxin, cytochrome P450, deoxycytidine kinase, tumor necrosis factor, a drug resistance protein, a tumor suppressor protein (e.g., p53, Rb, Wt-1, NF1, Von Hippel-Lindau (VHL), adenomatous polyposis coli (APC)), a peptide with immunomodulatory properties, a tolerogenic or immunogenic peptide or protein Tregitope or hCDR1, insulin, glucokinase, guanylate cyclase 2D (LCA-GUCY2D), Rab escort protein 1 (Choroideremia), LCA 5 (LCA-Lebercilin), ornithine ketoacid aminotransferase (Gyrate Atrophy), Retinoschisin 1 (X-linked Retinoschisis), USH1C (Usher's Syndrome 1C), X-linked retinitis pigmentosa GTPase (XLRP), MERTK (AR forms of RP: retinitis pigmentosa), DFNB1 (Connexin 26 deafness), ACHM 2, 3 and 4 (Achromatopsia), PKD-1 or PKD-2 (Polycystic kidney disease), TPP1, CLN2, a gene product implicated in lysosomal storage diseases (e.g., sulfatases, N-acetylglucosamine-1-phosphate transferase, cathepsin A, GM2-AP, NPC1, VPC2, a sphingolipid activator protein, one or more zinc finger nucleases for genome editing, or donor sequences used as repair templates for genome editing.

62. The method of any one of claims 2-58, wherein the heterologous polynucleotide encodes an inhibitory nucleic acid.

63. The method of any one of claims 2-58, wherein the inhibitory nucleic acid is selected from the group consisting of a siRNA, an antisense molecule, miRNA, RNAi, a ribozyme and a shRNA.

64. The method of claim 63, wherein the inhibitory nucleic acid binds to a pathogenic gene, a transcript of a pathogenic gene, or a gene transcript associated with a polynucleotide repeat disease, a huntingtin (HTT) gene, a gene associated with dentatorubropallidolusyan atropy (atrophin 1, ATN1), androgen receptor on the X chromosome in spinobulbar muscular atrophy, human Ataxin-1, -2, -3, and -7, Caᵥ2.1 P/Q voltage-dependent calcium channel is encoded by the (CACNA1A), TATA-binding protein, Ataxin 8 opposite strand, also known as ATXN8OS, Serine/threonine-protein phosphatase 2A 55 kDa regulatory subunit B beta isoform in spinocerebellar ataxia (type 1, 2, 3, 6, 7, 8, 12 17), FMR1 (fragile X mental retardation 1) in fragile X syndrome, FMR1 (fragile X mental retardation 1) in fragile X-associated tremor/ataxia syndrome, FMR1 (fragile X mental retardation 2) or AF4/FMR2 family member 2 in fragile XE mental retardation; Myotonin-protein kinase (MT-PK) in myotonic dystrophy; Frataxin in Friedreich's ataxia; a mutant of superoxide dismutase 1 (SOD1) gene in amyotrophic lateral sclerosis; a gene involved in pathogenesis of Parkinson's disease and/or Alzheimer's disease; apolipoprotein B (APOB) and proprotein convertase subtilisin/kexin type 9 (PCSK9), hypercoloesterolemia; HIV Tat, human immunodeficiency virus transactivator of transcription gene, in HIV infection; HIV TAR, HIV TAR, human immunodeficiency virus transactivator response element gene, in HIV infection; C-C chemokine receptor (CCR5) in HIV infection; Rous sarcoma virus (RSV) nucleocapsid protein in RSV infection, liver-specific microRNA (miR-122) in hepatitis C virus infection; p53, acute kidney injury or delayed graft function kidney transplant or kidney injury acute renal failure; protein kinase N3 (PKN3) in advance recurrent or metastatic solid malignancies; LMP2, LMP2 also known as proteasome subunit beta-type 9 (PSMB 9), metastatic melanoma; LMP7, also known as proteasome subunit beta-type 8 (PSMB 8), metastatic melanoma; MECL1 also known as proteasome subunit beta-type 10 (PSMB 10), metastatic melanoma; vascular endothelial growth factor (VEGF) in solid tumors; kinesin spindle protein in solid tumors, apoptosis suppressor B-cell CLL/lymphoma (BCL-2) in chronic myeloid leukemia; ribonucleotide reductase M2 (RRM2) in solid tumors; Furin in solid tumors; polo-like kinase 1 (PLK1) in liver tumors, diacylglycerol acyltransferase 1 (DGAT1) in hepatitis C infection, beta-catenin in familial adenomatous polyposis; beta2 adrenergic receptor, glaucoma; RTP801/Reddl also known as DAN damage-inducible transcript 4 protein, in diabetic macular oedma (DME) or age-related macular degeneration; vascular endothelial growth factor receptor I (VEGFR1) in age-related macular degeneration or choroidal neivascularization, caspase 2 in non-arteritic ischaemic optic neuropathy; Keratin 6A N17K mutant protein in pachyonychia congenital; influenza A virus genome/gene sequences in influenza infection; severe acute respiratory syndrome (SARS) coronavirus genome/gene sequences in SARS infection; respiratory syncytial virus genome/gene sequences in respiratory syncytial virus infection; Ebola filovirus genome/gene sequence in Ebola infection; hepatitis B and C virus genome/gene sequences in hepatitis B and C infection; herpes simplex virus (HSV) genome/gene sequences in HSV infection, coxsackievirus B3 genome/gene sequences in coxsackievirus B3 infection; silencing of a pathogenic allele of a gene (allele-specific silencing) like torsin A (TOR1A) in primary dystonia, pan-class I and HLA-allele specific in transplant; or mutant rhodopsin gene (RHO) in autosomal dominantly inherited retinitis pigmentosa (adRP).

65. The method of any one of claims 1-64, wherein the heterologous polynucleotide encodes a gene editing nuclease.

66. The method of claim 65, wherein the gene editing nuclease comprises a zinc finger nuclease (ZFN) or a transcription activator-like effector based nuclease (TALEN).

67. The method of any one of claims 1-64, wherein the heterologous polynucleotide encodes a functional Type II CRISPR-Cas9; and/or a guide RNA sequence; and/or a donor nucleic acid sequence for correction or replacement of a target gene.

68. The method of any one of claims 1-67, wherein step (a) and/or step (b) are performed two or more times.

69. The method of any one of claims 1-68, wherein the subject is a human.

70. An AAV binding antibody affinity matrix immobilized on a substrate disposed within a column, apparatus, chamber, device, filter, cartridge, tube having an inlet and an outlet for extracorporeal or intracorporeal removal or depletion of AAV binding antibodies from a blood product upon contact with the AAV binding antibody affinity matrix.

71. The AAV binding antibody affinity matrix of claim 70, comprising a naturally occurring or non-natural or synthetic intact AAV empty capsids.

72. The AAV binding antibody affinity matrix of claim 70, comprising an AAV VP1, VP2 and/or VP3 capsid protein or fragment thereof.

73. The AAV binding antibody affinity matrix of claim 70, wherein the AAV binding antibody affinity matrix comprises a naturally occurring or a non-natural or synthetic AAV VP1, VP2 and/or VP3 capsid protein.

74. The AAV binding antibody affinity matrix of claim 70, wherein the AAV binding antibody affinity matrix comprises a naturally occurring or a non-natural or synthetic AAV VP1, VP2 and/or VP3 capsid protein monomer.

75. The AAV binding antibody affinity matrix of claim 70, wherein the AAV binding antibody affinity matrix comprises a naturally occurring or a non-natural or synthetic AAV VP1, VP2 and/or VP3 capsid protein polymer.

76. The AAV binding antibody affinity matrix of claim 70, wherein the AAV binding antibody affinity matrix comprises AAV VP1, VP2 and/or VP3 capsid protein having 60% or more sequence identity to a naturally occurring or a non-natural or synthetic AAV capsid protein.

77. The AAV binding antibody affinity matrix of claim 70, wherein the AAV binding antibody affinity matrix comprises AAV VP1, VP2 and/or VP3 capsid protein having 60% or more sequence identity to an AAV VP1, VP2 and/or VP3 capsid protein selected from the group consisting of AAV1, AAV2, AAV3, AAV4, AAV5, AAV6, AAV7, AAV8, AAV9, AAV10, Rh10, Rh74, SEQ ID NO:1 and SEQ ID NO:2 VP1, VP2 and/or VP3 capsid proteins.

78. The AAV binding antibody affinity matrix of claim 70, wherein the AAV binding antibody affinity matrix comprises AAV VP1, VP2 and/or VP3 capsid protein having 60% or more sequence identity to SEQ ID NO:1 or SEQ ID NO:2.

79. The AAV binding antibody affinity matrix of claim 70, wherein the AAV binding antibody affinity matrix comprises an anti-idiotype antibody that binds to the AAV binding antibodies in the blood product.

80. The AAV binding antibody affinity matrix of claim 79, wherein the anti-idiotype antibody that binds to AAV binding antibodies binds to AAV1, AAV2, AAV3, AAV4, AAV5, AAV6, AAV7, AAV8, AAV9, AAV10, Rh10, Rh74, SEQ ID NO:1 and/or SEQ ID NO:2 capsid protein(s), or a derivative or amino acid substitution of AAV1, AAV2, AAV3, AAV4, AAV5, AAV6, AAV7, AAV8, AAV9, AAV10, Rh10, Rh74, SEQ ID NO:1 and/or SEQ ID NO:2 capsid protein(s).

81. The AAV binding antibody affinity matrix of claim 79, wherein the anti-idiotype antibody that binds to AAV binding antibodies is an antibody fragment.

82. The AAV binding antibody affinity matrix of claim 79, wherein the anti-idiotype antibody that binds to AAV binding antibodies is an IgG, IgA, IgM, IgE or IgD.

83. The AAV binding antibody affinity matrix of any of claims 70-82, wherein the AAV binding antibody affinity matrix is GMP grade.

84. The AAV binding antibody affinity matrix of any of claims 70-82, wherein leaching of the AAV binding antibody affinity matrix into a blood product obtained from a subject, if the blood product is reintroduced into the subject, does not substantially harm the subject.

85. The AAV binding antibody affinity matrix of any of claims 70-82, wherein the AAV binding antibody comprises an IgG, IgM, IgA or IgD that binds to AAV capsid protein.

86. The AAV binding antibody affinity matrix of any of claims 70-82, wherein the substrate and/or column, apparatus, chamber, device, filter, cartridge, or tube is configured from plastic or glass.

87. The AAV binding antibody affinity matrix of any of claims 70-82, wherein the AAV binding antibody affinity matrix, substrate and/or column, apparatus, chamber, device, filter, cartridge, tube are sterile.


SEQUENCE LISTING (2 sequences)

SEQ ID NO: 1 - protein, 738 aa, Adeno Associated Virus
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAAPSGVGPNTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 2 - protein, 736 aa, Adeno Associated Virus
MAADGYLPDWLEDNLSEGIREWWALQPGAPKPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRPL